(19) United States
(12) Patent Application Publication
(10) Pub. No.: US 2026/0049123 A1
(43) Pub. Date: Feb. 19, 2026

(54) BISPECIFIC ANTIBODIES PROMOTE NATURAL KILLER CELL-MEDIATED ELIMINATION OF HIV-1 RESERVOIR CELLS

(71) Applicant: The Johns Hopkins University, Baltimore, MD (US)

(72) Inventors: Nathan Louis Board, Baltimore, MD (US); Srona Sengupta, Baltimore, MD (US); Robert Siliciano, Baltimore, MD (US)

(21) Appl. No.: 19/036,463

(22) Filed: Jan. 24, 2025

Related U.S. Application Data
(60) Provisional application No. 63/621,708, filed on Jan. 17, 2024.

(51) Int. Cl. C07K 16/10; C07K 16/28

(52) U.S. Cl. CPC C07K 16/1063; C07K 16/283; C07K 2317/31; C07K 2317/569; C07K 2317/732

(57) ABSTRACT

The present invention relates to the field of virology. More specifically, the present invention provides compositions and methods useful for elimination of human immunodeficiency virus-1 (HIV-1) reservoir cells. In several embodiments, the single chain diabody binds human immunodeficiency virus 1 Envelope protein variable loops 1 and 2 (HIV-1 Env V1/V2) and human type III Fcγ receptor (CD16). In a specific embodiment, the scDb comprises (a) an anti-HIV-1 Env V1/V2 antigen binding fragment comprising a VH comprising or consisting of SEQ ID NO:1; (b) an anti-HIV Env V1/V2 antigen binding fragment comprising a VL comprising or consisting of SEQ ID NO:5; (c) an anti-CD16 antigen binding fragment comprising a VH comprising or consisting of SEQ ID NO:17; and (d) an anti-CD16 antigen binding fragment comprising a VL comprising or consisting of SEQ ID NO:21.

### CROSS-REFERENCE TO RELATED APPLICATIONS

This application claims the benefit of U.S. Provisional Application No. 63/621,708, filed Jan. 17, 2024, which is incorporated herein by reference in its entirety.

### FIELD OF THE INVENTION

The present invention relates to the field of virology. More specifically, the present invention provides compositions and methods useful for elimination of human immunodeficiency virus-1 (HIV-1) reservoir cells.

### REFERENCE TO AN ELECTRONIC SEQUENCE LISTING

The text of the computer readable sequence listing filed herewith, titled “P17715-02”, created Jan. 23, 2024, having a file size of 25,809 bytes, is hereby incorporated by reference in its entirety.

### BACKGROUND OF THE INVENTION

Antiretroviral therapy (ART) suppresses replication of HIV-1 (refs. 1,2) but is not curative owing to the persistence of long-lived, resting memory CD4+ T cells with latent HIV-1 proviruses^{3,4}. One approach to cure, known as ‘shock and kill’, involves selective reactivation of HIV-1 gene expression with latency reversing agents (LRAs) followed by immune-mediated clearance of infected cells^{5,6}. To date, LRA-based clinical interventions have not achieved substantial reduction in the latent reservoir despite observed increases in plasma and cell-associated viral RNA^{7,9}. A potential explanation is compromised function of cytolytic immune effector cells and inefficient elimination of cells expressing viral gene products¹⁰⁻¹².

One approach to improving cell-mediated elimination of HIV-1-infected cells involves bispecific antibodies. Bispecific antibodies can promote CD8+ T cell- or NK cell-mediated killing of tumor cells and have been used successfully for treatment of various cancers¹³. Thus far, bispecific antibody-based immunotherapies targeting HIV-1-infected cells have focused on engaging CD8+ T cells¹⁴⁻¹⁶ However, sustained exposure to HIV-1 antigens may promote CD8+ T cell exhaustion^{10,11} and several LRAs suppress the cytolytic potential of CD8+ T cells¹⁷⁻¹⁹. In addition, cytokine release syndrome has been reported in several clinical trials of T cell-engaging bispecific antibodies^{20,21}, giving rise to some safety concerns.

### SUMMARY OF THE INVENTION

The persistence of the long-lived HIV-1-infected cells comprising the latent reservoir is the main barrier to an effective cure. Novel therapeutics to enhance HIV-1-specific immune responses and clearance of infected cells will likely be necessary to achieve elimination of the latent reservoir.

In the present study, we propose the use of NK cells as effector cells in an alternative bispecific antibody approach. NK cells mediate robust antibody-dependent cellular cytotoxicity (ADCC) against HIV-1-infected cells, and elimination of HIV-1-infected cells through ADCC has been reported in protective vaccine responses and in suppression of viral replication in elite controllers⁶. Furthermore, NK cell-based approaches have not been reported to induce cytokine release syndrome. In the present study, we show that two bispecific antibodies targeting the HIV-1 Env and the human type III Fc receptor (CD16) enhance HIV-1 specific NK cell activation and cytolysis in vitro and NK cell-mediated reservoir clearance ex vivo. In addition, we found that one of the bispecific antibodies promotes elimination of HIV-1 infected cells in a mouse model of HIV-1 infection.

More specifically, the present inventors report two single-chain diabodies (scDbs) that target the HIV-1 envelope protein (Env) and the human type III Fc receptor (CD16). We show that the scDbs promoted robust and HIV-1-specific natural killer (NK) cell activation and NK cell-mediated lysis of infected cells. Cocultures of CD4+ T cells from people with HIV-1 on antiretroviral therapy (ART) with autologous NK cells and the scDbs resulted in marked elimination of reservoir cells that was dependent on latency reversal. Treatment of human interleukin-15 transgenic NSG mice with one of the scDbs after ART initiation enhanced NK cell activity and reduced reservoir size. Thus, HIV-1-specific scDbs merit further evaluation as potential therapeutics for clearance of the latent reservoir.

Thus, in one aspect, the present invention provides single chain diabodies. In several embodiments, the single chain diabody binds human immunodeficiency virus 1 Envelope protein variable loops 1 and 2 (HIV-1 Env V1/V2) and human type III Fcγ receptor (CD16). In a specific embodiment, the scDb comprises (a) an anti-HIV-1 Env V1/V2 antigen binding fragment comprising a VH comprising or consisting of SEQ ID NO:1; (b) an anti-HIV Env V1/V2 antigen binding fragment comprising a VL comprising or consisting of SEQ ID NO:5; (c) an anti-CD16 antigen binding fragment comprising a VH comprising or consisting of SEQ ID NO:17; and (d) an anti-CD16 antigen binding fragment comprising a VL comprising or consisting of SEQ ID NO:21.

In a more specific embodiment, a single-chain diabody that binds HIV-1 Env V1/V2 and CD16 comprises (a) an anti-HIV-1 Env V1/V2 antigen-binding fragment comprising a VH comprising CDRs 1, 2 and 3, wherein VH CDR1 comprises or consists of SEQ ID NO:2, VH CDR2 comprises or consists of SEQ ID NO:3, and VH CDR3 comprises or consists of SEQ ID NO:4; (b) an anti-HIV-1 Env V1/V2 antigen-binding fragment comprising a VL comprising CDRs 1, 2 and 3, wherein VL CDR1 comprises or consists of SEQ ID NO:6, VL CDR2 comprises or consists of SEQ ID NO:7, and VL CDR3 comprises or consists of SEQ ID NO:8; (c) an anti-CD16 antigen-binding fragment comprising a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and (d) an anti-CD16 antigen-binding fragment comprising a VL comprising CDRs 1, 2 and 3, wherein VL CDR1 comprises or consists of SEQ ID NO:22, VL CDR2 comprises or consists of SEQ ID NO:23, and VL CDR3 comprises or consists of SEQ ID NO:24.

In other embodiments, the present invention provides scDbs that bind HIV-1 CD4bs and CD16. In one embodiment, the scDb comprises (a) an anti-HIV-1 CD4bs antigen binding fragment comprising a VH comprising or consisting of SEQ ID NO:9; (b) an anti-HIV CD4bs antigen binding fragment comprising a VL comprising or consisting of SEQ ID NO:13; (c) an anti-CD16 antigen binding fragment comprising a VH comprising or consisting of SEQ ID NO:17; and (d) an anti-CD16 antigen binding fragment comprising a VL comprising or consisting of SEQ ID NO:21.

In a more specific embodiment, a single chain diabody that binds HIV-1 CD4bs and CD16 comprises (a) an anti-HIV-1 CD4bs antigen-binding fragment comprising a VH comprising CDRs 1, 2 and 3, wherein VH CDR1 comprises or consists of SEQ ID NO:10, VH CDR2 comprises or consists of SEQ ID NO:11, and VH CDR3 comprises or consists of SEQ ID NO:12; (b) an anti-HIV-1 CD4bs antigen-binding fragment comprising a VL comprising CDRs 1, 2 and 3, wherein VL CDR1 comprises or consists of SEQ ID NO:14, VL CDR2 comprises or consists of SEQ ID NO:15, and VL CDR3 comprises or consists of SEQ ID NO:16; (c) an anti-CD16 antigen-binding fragment comprising a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and (d) an anti-CD16 antigen-binding fragment comprising a VL comprising CDRs 1, 2 and 3, wherein VL CDR1 comprises or consists of SEQ ID NO:22, VL CDR2 comprises or consists of SEQ ID NO:23, and VL CDR3 comprises or consists of SEQ ID NO:24.

In another aspect, the present invention the present invention provides bispecific antibodies or antigen-binding fragments thereof that specifically binds HIV-1 Env V1/V2 and CD16. In a specific embodiment, the bispecific antibody or antigen-binding fragment there of comprises (a) a first antibody or antigen-binding fragment thereof that specifically binds HIV-1 Env V1/V2 comprising a VH comprising or consisting of SEQ ID NO:1 and a VL comprising or consisting of SEQ ID NO:5; and (b) a second antibody or antigen-binding fragment thereof that specifically binds CD16 comprising a VH comprising or consisting of SEQ ID NO:17 and a VL comprising or consisting of SEQ ID NO:21.

In a more specific embodiment, a bispecific antibody or antigen-binding fragment thereof that specifically binds HIV-1 Env V1/V2 and CD16 comprises (a) a first antibody or antigen-binding fragment thereof that specifically binds HIV-1 Env V1/V2 comprising (i) a heavy chain variable region (VH) comprising complementarity determining regions (CDRs) 1, 2 and 3, wherein VH CDR1 comprises or consists of SEQ ID NO:2, VH CDR2 comprises or consists of SEQ ID NO:3, and VH CDR3 comprises or consists of SEQ ID NO:4; and (ii) a light chain variable region (VL) comprising CDRs 1, 2 and 3, wherein VL CDR1 comprises or consists of SEQ ID NO:6, VL CDR2 comprises or consists of SEQ ID NO:7, and VL CDR comprises or consists of SEQ ID NO:8; and (b) a second antibody or antigen-binding fragment thereof that specifically binds CD16 comprising i) a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and (ii) a VL comprising CDRs 1, 2 and 3, wherein VL CDR1 comprises or consists of SEQ ID NO:22, VL CDR2 comprises or consists of SEQ ID NO:23, and VL CDR comprises or consists of SEQ ID NO:24.

In other embodiments, the present invention provides bispecific antibodies or antigen-binding fragments thereof that specifically bind HIV-1 Env CD4bs and CD16. In a specific embodiment, a bispecific antibody or antigen-binding fragment thereof comprises (a) a first antibody or antigen-binding fragment thereof that specifically binds HIV-1 CD4bs comprising a VH comprising or consisting of SEQ ID NO:9 and a VL comprising or consisting of SEQ ID NO:13; and (b) a second antibody or antigen-binding fragment thereof that specifically binds CD16 comprising a VH comprising or consisting of SEQ ID NO:17 and a VL comprising or consisting of SEQ ID NO:21.

In a more specific embodiment, a bispecific antibody or antigen-binding fragment thereof that specifically binds HIV-1 CD4 binding site (CD4bs) and CD16 comprises (a) a first antibody or antigen-binding fragment thereof that specifically binds HIV-1 CD4bs comprising (i) VH comprising CDRs 1, 2 and 3, wherein VH CDR1 comprises or consists of SEQ ID NO:10, VH CDR2 comprises or consists of SEQ ID NO:11, and VH CDR3 comprises or consists of SEQ ID NO:12; and (ii) a VL comprising CDRs 1, 2 and 3, wherein VL CDR1 comprises or consists of SEQ ID NO:14, VL CDR2 comprises or consists of SEQ ID NO:15, and VL CDR comprises or consists of SEQ ID NO:16; and (b) a second antibody or antigen-binding fragment thereof that specifically binds CD16 comprising i) a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and (ii) a VL comprising CDRs 1, 2 and 3, wherein VL CDR1 comprises or consists of SEQ ID NO:22, VL CDR2 comprises or consists of SEQ ID NO:23, and VL CDR comprises or consists of SEQ ID NO:24.

In some embodiments, a bispecific antibody or antibody-binding fragment thereof that specifically binds HIV-1 Env V1/V2 and CD16 comprises (a) a first antibody or antigen-binding fragment thereof that specifically binds HIV-1 Env V1/V2 comprising (i) VH comprising CDRs 1, 2 and 3, wherein VH CDR1 comprises or consists of SEQ ID NO:2, or SEQ ID NO:2 with a substitution at five or fewer amino acid positions, VH CDR2 comprises or consists of SEQ ID NO:3, or SEQ ID NO:3 with a substitution at seven or fewer amino acid positions, and VH CDR3 comprises or consists of SEQ ID NO:4, or SEQ ID NO:4 with a substitution at fifteen or fewer amino acid positions; and (ii) a VL comprising CDRs 1, 2 and 3, wherein VL CDR1 comprises or consists of SEQ ID NO:6, or SEQ ID NO:6 with a substitution at seven or fewer amino acid positions, VH CDR2 comprises or consists of SEQ ID NO:7, or SEQ ID NO:7 with a substitution at four or fewer amino acid positions, and VH CDR3 comprises or consists of SEQ ID NO:8, or SEQ ID NO:8 with a substitution at five or fewer amino acid positions; and (b) a second antibody or antigen-binding fragment thereof that specifically binds CD16 comprising (i) VH comprising CDRs 1, 2 and 3, wherein VH CDR1 comprises or consists of SEQ ID NO:18, or SEQ ID NO:18 with a substitution at three or fewer amino acid positions, VH CDR2 comprises or consists of SEQ ID NO:19, or SEQ ID NO:19 with a substitution at seven or fewer amino acid positions, and VH CDR3 comprises or consists of SEQ ID NO:20, or SEQ ID NO:20 with a substitution at four or fewer amino acid positions; and (ii) a VL comprising CDRs 1, 2 and 3, wherein VL CDR1 comprises or consists of SEQ ID NO:22, or SEQ ID NO:22 with a substitution at six or fewer amino acid positions, VH CDR2 comprises or consists of SEQ ID NO:23, or SEQ ID NO:23 with a substitution at four or fewer amino acid positions, and VH CDR3 comprises or consists of SEQ ID NO:24, or SEQ ID NO:24 with a substitution at six or fewer amino acid positions.

In certain embodiments, a bispecific antibody or antibody-binding fragment thereof that specifically binds HIV-1 CD4bs and CD16 comprises (a) a first antigen-binding fragment thereof that specifically binds HIV-1 Env V1/V2 comprising (i) VH comprising CDRs 1, 2 and 3, wherein VH CDR1 comprises or consists of SEQ ID NO:10, or SEQ ID NO:10 with a substitution at three or fewer amino acid positions, VH CDR2 comprises or consists of SEQ ID NO:11, or SEQ ID NO:11 with a substitution at nine or fewer amino acid positions, and VH CDR3 comprises or consists of SEQ ID NO:12, or SEQ ID NO:12 with a substitution at five or fewer amino acid positions; and (ii) a VL comprising CDRs 1, 2 and 3, wherein VL CDR1 comprises or consists of SEQ ID NO:14, or SEQ ID NO:14 with a substitution at four or fewer amino acid positions, VH CDR2 comprises or consists of SEQ ID NO:15, or SEQ ID NO:15 with a substitution at four or fewer amino acid positions, and VH CDR3 comprises or consists of SEQ ID NO:16, or SEQ ID NO:16 with a substitution at three or fewer amino acid positions; and (b) a second antigen-binding fragment thereof that specifically binds CD16 comprising (i) VH comprising CDRs 1, 2 and 3, wherein VH CDR1 comprises or consists of SEQ ID NO:18, or SEQ ID NO:18 with a substitution at three or fewer amino acid positions, VH CDR2 comprises or consists of SEQ ID NO:19, or SEQ ID NO:19 with a substitution at seven or fewer amino acid positions, and VH CDR3 comprises or consists of SEQ ID NO:20, or SEQ ID NO:20 with a substitution at four or fewer amino acid positions; and (ii) a VL comprising CDRs 1, 2 and 3, wherein VL CDR1 comprises or consists of SEQ ID NO:22, or SEQ ID NO:22 with a substitution at six or fewer amino acid positions, VH CDR2 comprises or consists of SEQ ID NO:23, or SEQ ID NO:23 with a substitution at four or fewer amino acid positions, and VH CDR3 comprises or consists of SEQ ID NO:24, or SEQ ID NO:24 with a substitution at six or fewer amino acid positions.

The present invention also provides bispecific antibodies or antigen-binding fragments thereof that specifically binds HIV-1 Env and CD16. In particular embodiments, the bispecific antibody or antigen-binding fragment thereof specifically binds HIV-1 Env V1/V2. In more particular embodiments, the bispecific antibody or antigen-binding fragment thereof specifically binds HIV-1 Env V1/V2 comprises a VH having at least 90% sequence identity to SEQ ID NO:2. In other embodiments, the bispecific antibody or antigen-binding fragment thereof specifically binds HIV-1 Env V1/V2 comprises a VL having at least 90% sequence identity to SEQ ID NO:5.

In other embodiments, the bispecific antibody or antigen-binding fragment thereof specifically binds HIV-1 CD4bs. In certain embodiments, the antibody or antigen-binding fragment thereof that specifically binds HIV-1 CD4bs comprises a VH having at least 90% sequence identity to SEQ ID NO:9. In other embodiments, the antibody or antigen-binding fragment thereof that specifically binds HIV-1 CD4bs comprises a VL having at least 90% sequence identity to SEQ ID NO:5.

In specific embodiments, the antibody or antigen-binding fragment thereof that specifically binds CD16 comprises a VH having at least 90% sequence identity to SEQ ID NO:17. In other specific embodiments, the antibody or antigen-binding fragment thereof that specifically binds CD16 comprises a VL having at least 90% sequence identity to SEQ ID NO:21.

In some embodiments of the present invention, the bispecific antibody or antigen-binding fragment thereof comprises tandem scFvs, diabody format, single-chain diabodies, tandem diabodies (TandAbs), dual-affinity retargeting molecules (DARTs), dock-and-lock (DNL), and nanobodies.

In another aspect, the present invention provides methods for treating HIV-1 in a patient. In some embodiments, the method comprises the step of administering to the patient an effective amount of a single chain diabody or a bispecific antibody or antigen-binding fragment thereof described herein.

### BRIEF DESCRIPTION OF THE FIGURES

FIG. 1A-1D. PG16-Db and 3BNC117-Db bind CD16 and Env. FIG. 1A: Schematic showing that the scDb consists of heavy- and light-chain variable regions of an Env-specific antibody and a CD16-specific antibody separated by glycine repeat linkers. ScDbs bind to CD16 expressed on the surface of cytolytic NK cells and to Env expressed on the surface of HIV-1-infected CD4+ T cells. FIG. 1B: Binding of H2-Db, PG16-Db and 3BNC117-Db to NK cells with or without blocking with a CD16− specific antibody (3G8) and to CD16− B cells. Data are reported as the percentage of cells that stained positive for the scDb (mean, s.d.) from three replicates. The significance was determined by two-way ANOVA followed by Dunnett's test for multiple comparisons, ****P<0.0001. FIG. 1C: Representative flow histograms showing the binding of H2-Db, PG16-Db and 3BNC117-Db to NK and B cells as in FIG. 1B at concentrations of 1,000 ng ml-1. Data are reported as fluorescence intensity. FIG. 1D: Binding of H2-Db, PG16-Db and 3BNC117-Db to HEK293T cells transfected with env from the HIV-1 isolates: 92/UG/092 (clade A), KNH1144 (clade A), BaL (clade B), BZ167 (clade B), NP1538 (clade B), 20635-4 (clade C), TZA246 (clade C) and D28630 (clade D). Data are reported as median fluorescence intensity (MFI) (mean, s.d.) from three replicates. The significance was determined by two-sided Student's t-tests against a theoretical mean of 0% reduction: *P<0.05, **P<0.01 for each tested env isolate at the indicated concentration.

FIG. 2A-B. PG16-Db and 3BNC117-Db induce polyfunctional HIV-1-specific NK cell responses. FIG. 2A: Expression of CD107a on CD3-CD56+NK cells cultured alone or with HIV-1-uninfected A3.01 cells or HIV-1-infected ACH-2 cells in the presence of H2-Db, PG16-Db and 3BNC117-Db. Data are reported as the percentage of cells that stained positive for CD107a (mean, s.d.) from three replicates. The significance was determined by two-way ANOVA followed by Dunnett's test for multiple comparisons: **P<0.01, ****P<0.0001 comparing the A3.01 with the ACH-2 conditions for both scDbs at the indicated concentrations. FIG. 2B: Representative flow histograms for CD107a expression on CD3-CD56+NK cells as in FIG. 2A. Data are reported as fluorescence intensity.

FIG. 3A-3B. PG16-Db and 3BNC117-Db promote NK cell-mediated elimination of HIV-1-infected cells. FIG. 3A: Representative flow plots showing the percentage of live GFP+CD4+ T cells after coculture with autologous NK cells alone or in the presence of 2,000 pM of H2-Db, PG16-Db, 3BNC117-Db, PG16-Ig or 3BNC117-Ig. FIG. 3B: Frequency of live GFP+CD4+ T cells (left) or p24 supernatant concentration (right) after coculture with autologous NK cells as in FIG. 3A relative to no scDb/IgG treatment. Data are reported as a percentage reduction (mean, s.d.) from three replicates. The significance was determined by two-way ANOVA followed by Dunnett's test for multiple comparisons: *P<0.05, ****P<0.0001 comparing the scDb with the IgG conditions for both PG16 and 3BNC117 at the indicated concentrations.

FIG. 4A-4C. PG16-Db and 3BNC117-Db eliminate cells harboring intact proviruses when paired with strong latency reversal. FIG. 4A: Frequency of intact provirus+CD4+ T cells after treatment with no LRA, bryostatin, AZD5582, romidepsin, SAHA, CD3+CD28 antibodies or PMA+I and coculture with autologous NK cells in the presence of H2-Db, PG16-Db, 3BNC117-Db or PG16-Db+3BNC117-Db. Data are reported as intact HIV-1 copies per million viable CD4+ T cells (mean, s.d.) from eight replicates. The significance was determined by two-way ANOVA followed by Dunnett's test for multiple comparisons: *P<0.05, **P<0.01, ***P<0.001, ****P<0.0001. FIG. 4B: Expression of caRNA after treatment with LRAs as in FIG. 4A. Data are reported as caRNA copies per million CD4+ T cells (mean, s.d.) from three replicates. The significance was determined by one-way ANOVA followed by Dunnett's test for multiple comparisons: ***P<0.001, ****P<0.0001. FIG. 4C: Correlation between percentage reduction in intact copies and mean fold-change in caRNA. Spearman's r value is shown.

FIG. 5A-5B. PG16-Db and 3BNC117-Db eliminate cells harboring intact and inducible proviruses. FIG. 5A: Frequency of intact provirus+CD4+ T cells after treatment with PMA+I and coculture with autologous NK cells in the presence of H2-Db, PG16-Db, 3BNC117-Db or PG16-Db+3BNC117-Db. Data are reported as intact HIV-1 copies per million viable CD4+ T cells (mean, s.d.) from eight replicates. The significance was determined by one-way ANOVA followed by Dunnett's test for multiple comparisons: *P<0.05, **P<0.01, ***P<0.001, ****P<0.0001. FIG. 5B: Average frequencies of intact provirus+CD4+ T cells (top left), 5′-deleted provirus+CD4+ T cells (bottom far left) and 3′-deleted/hypermutated provirus+CD4+ T cells (bottom middle left) after treatment with PMA+I and coculture with autologous NK cells as in FIG. 5A (mean, s.d.) across all 11 participants assessed. The significance was determined by one-way ANOVA followed by Dunnett's test for multiple comparisons: *P<0.05, **P<0.01. Average IUPM viable CD4+ T cells (bottom middle right) after treatment with PMA+I and coculture with autologous NK cells as in FIG. 5A (mean, s.d.) across six participants assessed. The significance was determined by paired, two-sided Student's t-test: **P<0.01. Average frequency of intact provirus+CD4+ T cells (top middle) or average IUPM viable CD4+ T cells (bottom far right) relative to H2-Db. Data reported as a percentage reduction (mean, s.d.) from eleven or six participants, respectively. The significance was determined by Student's t-tests against a theoretical mean of 0% reductions: ***P<0.001, ****P<0.0001.

FIG. 6A-6D. 3BNC117-Db induces NK cell activity and elimination of infected cells in hIL-15TgNSG mice. FIG. 6A: Frequencies of human CD4+ T cells and CD3-CD56+NK cells in the peripheral blood at 12 weeks posttransfer of human fetal liver and thymus tissues into hIL-15TgNSG mice. Mice were randomly assigned to two groups corresponding to treatment with H2-Db (n=9) or 3BNC117-Db (n=9) at day 33 post-infection. Data are reported as the percentage of PBMCs that expressed the indicated markers (mean, s.d.). FIG. 6B: Schematic showing hIL-15TgNSG mice infected with HIVSUMA at 12 weeks postreconstitution as in FIG. 6A, administered ART from day 14 to day 49 post-infection, injected intraperitoneally with H2-Db or 3BNC117-Db daily between day 33 and day 43 post-infection, sampled for peripheral blood on days 0, 7, 14, 21, 28, 35, 42 and 49 post-infection (small black arrows) and euthanized at day 49 post-infection for collection of splenic tissue. FIG. 6C: HIV-1 RNA copies per ml of plasma (far left), percentage of CD4+ T cells (middle left), percentage of CD3-CD56+ lymphocytes (middle), percentage of HLA-DR+NK cells (middle right) and percentage of CD57+NK cells (far right) from day 0 to day 49 post-infection of hIL-15TgNSG mice. Data are shown as both individual values (top) and average values (bottom) for both the H2-Db- and 3BNC117-Db-treated mice (mean, s.d.): H2-Db (n=9), 3BNC117-Db (n=9). The significance was determined by two-way ANOVA followed by Sidak's test for multiple comparisons: *P<0.05, **P<0.01, ***P<0.001, ****P<0.0001. FIG. 6D: Frequencies of intact provirus+CD4+ T cells, 5′-deleted provirus+CD4+ T cells and 3′-deleted/hypermutated provirus+CD4+ T cells in hIL-15TgNSG mice at day 49 post-infection. Data are reported as HIV-1 copies per million human CD4+ T cells (mean, s.d.): H2-Db (n=9), 3BNC117-Db (n=9). The significance was determined by two-way ANOVA followed by Sidak's test for multiple comparisons: ***P<0.001, ****P<0.0001. Comparisons between intact copies per million human CD4+ T cells percentage of CD3-CD56+ lymphocytes (middle left), percentage of HLA-DR+NK cells (middle right) and percentage of CD57+NK cells (far right) posttreatment (day 42).

FIG. 7A-7D. H2-Db, PG16-Db, and 3BNC117-Db bind both CD16a recombinant protein and cell surface CD16a. FIG. 7A: Binding of H2-Db, PG16-Db, 3BNC117-Db to immobilized CD16a recombinant proteins. Both CD16 variants are identical except for a single amino acid substitution at position 158 (F/V). Data reported as the optical density at 450 nm (OD450) (mean, SD) from three replicates and were fitted to a dose-response nonlinear regression model. FIG. 7B: Calculated EC50 values for the fitted nonlinear regression model from a reported in ng/mL. FIG. 7C: Binding of H2-Db, PG16-Db, and 3BNC117-Db to peripheral blood lymphocytes, CD3-CD56+NK cells, CD3+ T cells, and CD19+ B cells with or without blocking with a CD16-specific antibody (3G8). PBMCs were stained with 1000 ng/mL of H2-Db, PG16-Db, or 3BNC117-Db. Data reported as the percentage of cells that stained positive for the indicated scDb (mean, SD) from three replicates. FIG. 7D: Expression of CD16 on peripheral blood lymphocytes, CD3-CD56+NK cells, CD3+ T cells, and CD19+ B cells. Data reported as the percentage of CD16+ cells (mean, SD) from three replicates.

FIG. 8A-8D. PG16-Db and 3BNC117-Db promote elimination of Env+ cells. FIG. 8A: Binding of PG16-Ig and 3BNC117-Ig to NL4.3-ΔNef-eGFP-infected CD4+ T cells. Data reported as the percentage of hIgG Fc+ CD4+ T cells after preincubation of the cells with 20 g/mL of PG16-Ig, 3BNC117-Ig, or an equimolar combination of PG16-Ig+3BNC117-Ig (mean, SD) from three replicates. FIG. 8B: Representative flow histograms showing binding of PG16-Ig, 3BNC117-Ig, and PG16-Ig+3BNC117-Ig to GFP+CD4+ T cells. All conditions shown are after pre-incubation of the infected CD4+ T cells with 20 g/mL of the corresponding IgG. Data reported as fluorescence intensity. FIG. 8C: CD4+ T cells infected with NL4.3-ΔNef-eGFP were co-cultured with autologous NK cells in the presence of H2-Db, PG16-Db, 3BNC117-Db, PG16-Ig, or 3BNC117-Ig at 2000 pM. Data reported as the frequency of viable GFP+ cells relative to no scDb/IgG treatment (percent reduction, mean, SD) from three replicates. Significance determined by one-way ANOVA followed by Dunnett's test for multiple comparisons, ****P<0.0001. FIG. 8D: CD4+ T cells infected with NL4.3 were co-cultured with autologous NK cells in the presence of H2-Db, PG16-Db, 3BNC117-Db, PG16-Ig, or 3BNC117-Ig. Data reported as the frequency of viable p24+ cells relative to no scDb/IgG treatment (percent reduction, mean, SD) from three replicates. Significance determined by one-way ANOVA followed by Dunnett's test for multiple comparisons, ****P<0.0001.

FIG. 9. Efficacy of scDb-mediated reduction intact proviruses+CD4+ T cells cannot be attributed to NK cell phenotype. Expression of CD16, NKG2D, Siglec-7, CD57, and PD-1 on CD3-CD56+NK cells. Participants were categorized into two subgroups, high response (HR, n=4) and low response (LR, n=4), based on whether that individual had an above or below average reduction in intact provirus+ cells with PG16-Db+3BNC117-Db. HIV-participants were included for comparison (n=3). Data reported as the percentage of cells that stained positive for the indicated marker (mean, SD). Correlations between percent reduction in intact copies and percentage of CD3-CD56+NK cells that stained positive for the indicated markers. Spearman's r value shown.

FIG. 10. PG16-Db and 3BNC117-Db eliminate CD4+ T cells harboring inducible and replication-competent proviruses. CD4+ T cells were treated with PMA+I and co-culture with autologous NK cells in the presence of PG16-Db+3BNC117-Db. After co-culture, the viable CD4+ T cells were plated at limiting dilution for quantitative viral outgrowth assays (QVOAs). Concentrations of p24 was measured in the culture supernatants on day 14. Data reported as infectious units per million (IUPM) viable CD4+ T cells (maximum-likelihood estimate, 95% confidence interval upper bound).

FIG. 11A-11B. PG16-Db and 3BNC117-Db do not eliminate cells harboring defective proviruses ex vivo. Frequencies of CD4+ T cells harboring FIG. 11A, 5′ deleted or FIG. 11B, 3′ deleted or hypermutated proviruses after treatment with PMA+I and co-culture with autologous NK cells in the presence of PG16-Db, 3BNC117-Db, or PG16-Db+3BNC117-Db. Data reported as defective HIV-1 copies per million viable CD4+ T cells (mean, SD) from eight replicates.

FIG. 12. H2-Db and 3BNC117-Db plasma pharmacokinetics in hIL-15TgNSG mice. The mice received a single 200 g intraperitoneal injection of H2-Db or 3BNC117-Db and scDb plasma concentrations were assessed at 0, 2, 4, 8, 12, 24, and 48 hours post-injection. Plasma scDb concentrations are shown for the individual mice (top) or the average values (bottom) (mean, SD, 5 mice per group). Data were fitted to a single-phase exponential decay nonlinear regression model and predicted plasma half-lives are indicated.

FIG. 13. Tolerability of H2-Db and 3BNC117-Db in hIL-15TgNSG mice. Body weight of the mice reported in grams. Data reported as weights of individual mice over time (top) and weights prior to infection, at the time of ART initiation, and before and after scDb treatment (bottom) (mean, SD). H2-Db (n=9); 3BNC117-Db (n=9). No significant differences between H2-Db- and 3BNC117-Db-treated mice at any time point or significant changes in body weights compared to day 0 within either group at any time point were determined by two-way ANOVA followed by Sidak's test for multiple comparisons.

FIG. 14. Additional measures of H2-Db and 3BNC117-Dbmediated activity in hIL-15TgNSG mice. Plasma viremia and peripheral blood NK cell activity in the hIL-15TgNSG mice prior to infection, at the time of ART initiation, and before and after H2-Db and 3BNC117-Db treatment. Data reported as HIV-1 RNA copies/mL plasma (top left), percentage of CD3-CD56+ lymphocytes (top right), percentage of HLA-DR+NK cells (bottom left), and percentage of CD57+NK cells (mean, SD). H2-Db (n=9); 3BNC117-Db (n=9). Significance determined by two-way ANOVA followed by Sidak's test for multiple comparisons, *P<0.05, **P<0.01, ****P<0.0001.

FIG. 15. H2-Db and 3BNC117-Db do not induce global T cell activation in hIL-15TgNSG mice. Frequency of peripheral blood CD4+(left) and CD8+(right) T cells that expressed HLA-DR and CD38 over time in the hIL-15TgNSG mice. Data shown for individual mice (top) and average percentages (bottom) (mean, SD). H2-Db (n=9); 3BNC117-Db (n=9). No significant differences between H2-Db- and 3BNC117-Db-treated mice at any time point or significant changes in frequencies of HLA-DR+CD38+ T cells compared to day 0 within either group at any time point were determined by two-way ANOVA followed by Sidak's test for multiple comparisons.

FIG. 16. Comparisons between the frequencies of defective provirus+CD4+ T cells and NK cell activity in H2-Db- and 3BNC117-Db-treated hIL-15TgNSG mice. Data reported as the percentage of CD3-CD56+ lymphocytes (left), percentage of HLA-DR+NK cells (middle), and percentage of CD57+NK cells (right) post-treatment (day 42) compared to the frequencies of CD4+ T cells harboring 5′ deleted (top) or 3′ deleted or hypermutated proviruses (bottom). H2-Db (n=9); 3BNC117-Db (n=9).

FIG. 17. H2-Db, PG16-Db, and 3BNC117-Db purity after size-exclusion chromatography. Representative analytical chromatogram of the purified H2-Db (left). Data reported as absorbance at 280 nm over retention time. The peak retention time is indicated above the chromatogram. SDS-PAGE analysis of H2-Db, PG16-Db, and 3BNC117-Db (right). Expected molecular weights of H2-Db, PG16-Db, and 3BNC117-Db are 52 kDa, 54 kDa, and 53 kDa respectively.

FIG. 18. Expression of Env on transfected HEK293T cells. Binding of PG16-Ig and 3BNC117-Ig to HEK293T cells transfected with env from cells transfected with env from the HIV-1 isolates: 92/UG/092 (clade A), KNH1144 (clade A), BaL (clade B), BZ167 (clade B), NP1538 (clade B), 20635-4 (clade C), TZA246 (clade C), and D28630 (clade D). All conditions shown are after pre-incubation of the transfected HEK293T cells with 20 g/mL of the corresponding IgG. Data reported as median fluorescence intensity (MFI) (mean, SD) from three replicates.

FIG. 19. PG16-Db and 3BNC117-Db elicit enhanced NK cell degranulation relative to parental IgGs. Expression of CD107a measured on CD3− CD56+NK cells either alone or co-cultured with uninfected A3.01 cells or HIV-1-infected ACH-2 cells in the presence of the H2-Db, PG16-Db, 3BNC117-Db, PG16-Ig, or 3BNC117-Ig at 2000 pM. Data reported as the percentage of CD107a+ NK cells (mean, SD) from three replicates. Significance determined by two-way ANOVA followed by Dunnett's test for multiple comparisons, ****P<0.0001.

FIG. 20. Gating strategy for the NK surface CD16 binding assay. NK cells: lymphocyte size (FSC-H, SSC-H)>single cells (FSC-H, FSC-A)>live (eFlour780)>CD3-(BV605)/CD56+(PE/Cy5)>scDb+(AF488). B cells: lymphocyte size (FSC-H, SSC-H)>single cells (FSC-H, FSC-A)>live (eFlour780)>CD19+(BV421)>scDb+(AF488).

FIG. 21. Gating strategy for the surface Env binding assay. HEK293T size (FSC-H, SSC-H)>single cells (FSC-H, FSC-A)>live (eFlour780).

FIG. 22. Gating strategy for the NK activation co-culture. Lymphocyte size (FSC-H, SSC-H)>single cells (FSC-H, FSC-A)>live (eFlour780)>CD3− (BV605)/CD56+(FITC)>CD107a+(BV421).

FIG. 23. Gating strategy for the in vitro infected cell elimination co-culture. Lymphocyte size (FSC-H, SSC-H)>single cells (FSC-H, FSC-A)>CD3+(BV605)/CD56-(PE/Cy5)>live (eFlour780)/GFP+.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “epitope” refers to a portion of an antigen that specifically binds to an antibody or antigen-binding fragment or domain thereof. Epitopes can, for example, consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

The term “antibody” means an immunoglobulin molecule that recognizes and specifically binds to a target, such as a protein (e.g., HIV-1 Env protein or or CD16), polypeptide, peptide, carbohydrate, polynucleotide, lipid, or combinations of the foregoing through at least one antigen recognition site within the variable region of the immunoglobulin molecule. A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CHI, CH2, and CH3. Each light chain is comprised of a “light chain variable region” or “light chain variable domain” (abbreviated herein as VL) and a light chain constant region. The light chain constant region is comprised of one domain, CI. The VH and VL regions can be further subdivided into regions of hypervariability, termed Complementarity Determining Regions (CDR), interspersed with regions that are more conserved, termed framework regions (FRs). Each VH and VL region is composed of three CDRs and four FRs, arranged from amino-terminus to carboxy-terminus in the following order: FRI, CDRI, FR2, CDR2, FR3, CDR3, FR4. The variable regions of the heavy and light chains contain a binding domain that interacts with an antigen. As used herein, the term “antibody” encompasses intact poly clonal antibodies, intact monoclonal antibodies, antibody fragments (such as Fab, Fab′, F(ab′)2, Fd, Facb, and Fv fragments), single chain Fv (scFv), minibodies (e.g., sc(Fv)2, diabody), multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, radioisotopes, small molecule drugs, polypeptides, etc.

The term “isolated antibody” refers to an antibody that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody is purified (1) to greater than 95% by weight of antibody as determined by, for example, the Lowry method, and including more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, isolated antibody will be prepared by at least one purification step.

The term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human.

The term “antigen-binding fragment” or “antigen-binding domain” refers to a portion of an intact antibody and refers to the antigenic determining variable regions of an intact antibody. It is known in the art that the antigen binding function of an antibody can be performed by fragments of a full-length antibody. Examples of antigen-binding antibody fragments include, but are not limited to Fab, Fab′, F(ab′)2, Facb, Fd, and Fv fragments, linear antibodies, single chain antibodies, and multi-specific antibodies formed from antibody fragments. In some instances, antibody fragments may be prepared by proteolytic digestion of intact or whole antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced. The term “F(ab′)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced. The term “Fv” refers to an antibody fragment that consists of one NH and one N domain held together by noncovalent interactions.

The term “single-chain Fv” or “scFv” as used herein refers to antibody fragments comprising the VH and VL domains of antibody, wherein these domains are present in a single polypeptide chain. In particular embodiments, the Fv polypeptide further comprises a polypeptide linker between the VH and VL domains which enables the scFv to form the desired structure for antigen binding.

The term “diabody” as used herein refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites.

The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2, 3, 4, 5, 6, 7, 8, 9, 10, 11, 12, 13, 14, 15, 16, 17, 18, 19, 20, 25, 30, 50, or 100 VH domains. The tandem of VH can be produced by joining the encoding genes of each VH domain in a desired order using recombinant methods with or without a linker (e.g., a synthetic linker) that enables them to be made as a single protein. In one aspect, the VHI domains in the tandem, alone or in combination with VL domains of the same antibody, retain the binding specificity of the original antibody. The N-terminus of the first VHI domain in the tandem is defined as the N-terminus of the tandem, while the C-terminus of the last VH domain in the tandem is defined as the C-terminus of the tandem.

The term “a tandem of V L domains (or VLs)” as used herein refers to a string of VL domains, consisting of multiple numbers of identical VL domains of an antibody. Each of the VL domains, except the last one at the end of the tandem, has its C-terminus connected to the N-terminus of another VH with or without a linker. A tandem has at least 2, 3,4, 5, 6, 7, 8, 9, 10, 11, 12, 13, 14, 15, 16, 17, 18, 19, 20, 25, 30, 50, or 100 VL domains. The tandem of VL can be produced by joining the encoding gene of each VL domain in a desired order using recombinant methods with or without a linker (e.g., a synthetic linker) that enables them to be made as a single protein. In particular embodiments, the VL domains in the tandem, alone or in combination with VH domains of the same antibody, retain the binding specificity of the original antibodies. The N-terminus of the first VL domain in the tandem is defined as the N-terminus of the tandem, while the C-terminus of the last VL domain in the tandem is defined as the C-terminus of the tandem.

A “modification” or “mutation” of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more amino acids adjacent to said residue/position, and deletion of said residue/position. An “amino acid substitution,” or variation thereof, refers to the replacement of an existing amino acid residue in a predetermined (starting) amino acid sequence with a different amino acid residue. Generally and preferably, the modification results in alteration in at least one physicobiochemical activity of the variant polypeptide compared to a polypeptide comprising the starting (or “wild type”) amino acid sequence. For example, in the case of an antibody, a physicobiochemical activity that is altered can be binding affinity, binding capability and/or binding effect upon a target molecule.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the phrase “conservative sequence modifications” are used to refer to amino acid modifications that do not significantly affect or alter the binding characteristics of the antibody or the antibody-like molecule containing the amino acid sequence.

The term “% identical” between two polypeptide (or polynucleotide) sequences refers to the number of identical matched positions shared by the sequences over a comparison window, considering additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site. Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2 (ClustalX is a version of the ClustalW2 program ported to the Windows environment). Another suitable program is MUSCLE. ClustalW2 and MUSCL.

### II. Bispecific Antibodies Against Human Immunodeficiency Virus-1 (HIV-1) Envelope (Env) Protein and Type III Fcγ Receptor (CD16)

Bispecific antibodies are antibodies that have binding specificities for at least two different epitopes. Such antibodies may combine a HIV-1 Env protein site with a binding site for another protein, such as CD16. Bispecific antibodies can be prepared as full length antibodies or low molecular weight forms thereof (e.g., F(ab′) 2 bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

In some embodiments, bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

In certain embodiments, a bispecific antibody of the present invention can be in any appropriate format which includes at least one VH and at least one VL. For example, a VH and a VL can be in any appropriate orientation. In some cases, a VH can be N-terminal to the VL. In some cases, a VH can be C-terminal to the VL. In some cases, a linker amino acid sequence can be positioned between the VH and VL.

In particular embodiments, when a bispecific molecule comprises tandem scFvs, the tandem scFvs can be in any appropriate orientation. Examples of tandem scFv orientations including scFv-A and scFv-B include, without limitation, VLA-LL-VHA-SL-VLB-LL-VHB, VLA-LL-VHA-SL-VHB-LL-VLB, VHA-LL-VLA-SL-VLB-LL-VHB, VHA-LL-VLA-SL-VHB-LL-VLB, VLB-LL-VHB-SL-VLA-LL-VHA, VLB-LL-VHB-SL-VHA-LL-VLA, VHB-LL-VLB-SL-VLA-LL-VHA, and VHB-II-VLB-SL-VHA-LL-VLA, where SL is a short linker and LL is a long linker. A short linker can be from about 3 amino acids to about 10 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination.

In other embodiments, when a bispecific molecule is a diabody, the diabody can be in any appropriate orientation. Examples of diabody orientations including scFv-A and scFv-B include, without limitation. VLA-SL-VHB and VLB-SL-VHA, VLA-SL-VLB and VHB-SL-VHA, VHA-SL-VLB and VHB-SL-VLA, VLB-SL-VIA and VLA-SL-VHB, VLB-SL-VLA and VHA-SL-VHB, and VHB-SL-VLA and VHA-SL-VLB, where SL is a short linker. A short linker can be from about 3 amino acids to about 10 amino acids in length. A short linker can include any appropriate amino acids (e.g., glycines and serines) in any appropriate combination.

In some embodiments, when a bispecific antibody or antigen-binding fragment/domain is a single-chain diabody (scDb), the scDb can be in any appropriate orientation. Examples of scDb orientations including scFv-A and scFv-3 include, without limitation, VLA-SL-VHB-LL-VLB-SL-VHA, VHA-SL-VLB-LL-VHB-SL-VLA, VLA-SL-VLB-LL-VHB-SL-VHA, VHA-SL-VHB-LL-VLB-SL-VLA, VLB-SL-VHA-LL-VLA-SL-VHB, VHB-SL-VLA-LL-VHA-SL-VLB, VLB-SL-VLA-LL-VHA-SL-VHB, and VHB-SL-VHA-LL-VLA-SL-VLB, where SL is a short linker and LL is a long linker. A short linker can be from about 3 amino acids to about 10 amino acids in length. A short linker can include any appropriate amino acids (e.g., glycines and serines) in any appropriate combination. A long linker can be from about 10 amino acids to about 25 amino acids in length, A long linker can include any appropriate amino acids (e.g., glycines and serines) in any appropriate combination.

In certain embodiments, when a bispecific molecule is a scFv-Fc, the scFv-Fc can be in any appropriate orientation. Examples of scFv-Fc orientations including scFv-Fc-A. scFv-Fc-B, and an Fc domain include, without limitation, VLA-LL-VHA-hinge-Fc and VLB-LL-VHB-hinge-Fc, VHA-LL-VLA-hinge-Fc and VHB-LL-VLB-hinge-Fc, VLA-LL-VHA-hinge-Fc and VHB-LL-VLB-hinge-Fc, VHA-LL-VLA-hinge-Fc and VLB-LL-VHB-hinge-Fc, where LL is a long linker. A long linker can be from about 10 amino acids to about 25 amino acids in length. A long linker can include any appropriate amino acids (e.g., glycines and serines) in any appropriate combination. In some cases, an Fc domain in a scFv-Fc can include one or more modifications to increase heterodimerization and/or to decrease homodimerization of the scFv-Fc. In some cases, an Fc domain in a scFv-Fc can exclude a hinge domain. In some cases, an Fc domain in a scFv-Fc can be at the N-terminus of the scFv.

In other embodiments, when a bispecific molecule is a bispecific single-chain Fc, the bispecific single-chain Fc can be in any appropriate orientation. Examples of bispecific single-chain Fc orientations include, without limitation, VLA-LL-VHA-SL-VHB-LL-VLB-SL-hinge-CH2-CH3-LL-hinge-CH2-CH3, VLA-LL-VHA-SL-VLB-LL-VHB-SL-hinge-CH2-CH3-LL-hinge-CH2-CH3, VHA-LL-VLA-SL-VLB-LL-VHB-SL-hinge-CH2-CH3-LL-hinge-CH2-CH3, VIA-LL-VLA-SL-VHB-LL-VLB-SL-hinge-CH2-CH3-LL-hinge-CH2-CH3, and VLA-SL-VHB-LL-VLB-VHA-SL-hinge-CH2-CH3-LL-hinge-CH2-CH-3, where SL is a short linker and LL is a long linker. A short linker can be from about 3 amino acids to about 8 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination. Any appropriate Fc domain can be used in a bispecific single-chain Fe. In some cases, an Fc domain can include an amino acid sequence derived from an IgG (e.g., a natural IgG). In some cases, an Fc domain can include an amino acid sequence that includes one or more modifications (e.g., one or more modifications to increase stability of the molecule and/or to increase or decrease binding to one or more Fc receptors). In some cases, an Fc domain that can be used in a bispecific single-chain Fc can exclude a hinge domain. In some cases, an Fc domain that can be used in a bispecific single-chain Fc can be at the N-terminus of the scFvs. In some cases, an Fc domain that can be used in a bispecific single-chain Fc can be as described elsewhere (see, e.g., International Patent Application Publication No. WO 2017/134134 A1 at, for example, SEQ ID NOs: 25-32; and International Patent Application Publication No. WO 2017/134158 A1 at, for example, Table 38; and SEQ ID NOs: 25-32).

In some embodiments, a bispecific antibody of the present invention comprising a first antigen-binding domain that specifically binds HIV-1 Env protein further comprises a second antigen-binding domain or fragment that specific binds an effector cell. Examples of effector cells include, without limitation, natural killer (N K) cells, natural killer T (NKT) cells, T cells, B cells, plasma cells, macrophages, monocytes, microglia, dendritic cells, neutrophils, fibroblasts, and mast cells. Examples of antigens present on effector cells include, without limitation, CD3, CD4, CD8, CD28, NKG2D, PD-1, CTLA-4, 4-IBB, OX40, ICOS, CD27, Fc receptors (e.g., CD16a), and any other effector cell surface receptors. In some embodiments, a molecule described herein can include a first antigen-binding domain that can bind to a modified peptide described herein and a second antigen-binding domain that can bind to an antigen present on a NK cell (e.g., CD16a or NKG2D).

The bispecific antibodies or antigen-binding fragments thereof of this disclosure specifically bind to HIV-1 Env and CD16. In specific embodiments, these antibodies or antigen-binding fragments specifically bind to HIV-1 Env (e.g., V1/V2 or CD4bs) and CD16a. “Specifically binds” as used herein means that the antibody or antigen-binding fragment preferentially binds its target(s) over other proteins. In certain instances, the anti-HIV-1 Env/anti-CD16 bispecific antibodies of the disclosure have a higher affinity for HIV-1 Env/CD16 than for other proteins. Anti-HIV-1 Env/anti-CD16 bispecific antibodies that specifically bind HIV-1 Env/CD16 may have a binding affinity for human HIV-1 Env/CD16 of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. Methods of measuring the binding affinity of an antibody are well known in the art and include Surface Plasmon Resonance (SPR) (Morton and Myszka “Kinetic analysis of macromolecular interactions using surface plasmon resonance biosensors” Methods in Enzymology (1998) 295, 268-294), Bio-Layer Interferometry, (Abdiche et al “Determining Kinetics and Affinities of Protein Interactions Using a Parallel Real-time Label-free Biosensor, the Octet” Analytical Biochemistry (2008) 377, 209-217), Kinetic Exclusion Assay (KinExA) (Darling and Brault “Kinetic exclusion assay technology: characterization of molecular interactions” Assay and Drug Dev Tech (2004) 2, 647-657), isothermal calorimetry (Pierce et al “Isothermal Titration Calorimetry of Protein-Protein Interactions” Methods (1999) 19, 213-221) and analytical ultracentrifugation (Lebowitz et al “Modern analytical ultracentrifugation in protein science: A tutorial review” Protein Science (2002), 11:2067-2079).

In specific embodiments, provided herein is an anti-HIV-1 Env V1/V2 antibody or antigen-binding fragment thereof comprising a heavy chain variable domain comprising the amino acid sequence of SEQ ID NO:1 and comprising a light chain variable domain comprising the amino acid sequence of SEQ ID NO:5. See Table 1.

In particular embodiments, provided herein is an anti-HIV-I Env V1/V2 antibody or antigen-binding fragment thereof comprising a heavy chain variable region and a light chain variable region, wherein the heavy chain variable region comprises (i) CDR-H1 comprising the amino acid sequence of SEQ ID NO:2, (ii) CDR-H2 comprising the amino acid sequence of SEQ ID NO:3, and (iii) CDR-1-3 comprising the amino acid sequence of SEQ ID NO:4; and/or wherein the light chain variable region comprises (i) CDR-L1 comprising the amino acid sequence of SEQ ID NO:6, (ii) CDR-L2 comprising the amino acid sequence of SEQ ID NO:7, and (iii) CDR-L3 comprising the amino acid sequence of SEQ ID NO:8 wherein the CDRs of the anti-HIV 1 Env V1/V2 antibody are defined by the Kabat numbering scheme. See Table 1.

In specific embodiments, provided herein is an anti-HIV-1 Env CD4bs antibody or antigen-binding fragment thereof comprising a heavy chain variable domain comprising the amino acid sequence of SEQ ID NO:9 and comprising a light chain variable domain comprising the amino acid sequence of SEQ ID NO:13. See Table 1.

In particular embodiments, provided herein is an anti-HIV-1 Env CD4bs antibody or antigen-binding fragment thereof comprising a heavy chain variable region and a light chain variable region, wherein the heavy chain variable region comprises (i) CDR-H1 comprising the amino acid sequence of SEQ ID NO:10, (ii) CDR-H2 comprising the amino acid sequence of SEQ ID NO:11, and (iii) CDR-L3 comprising the amino acid sequence of SEQ ID NO:12; and/or wherein the light chain variable region comprises (i) CDR-L1 comprising the amino acid sequence of SEQ ID NO:14, (ii) CDR-L2 comprising the amino acid sequence of SEQ ID NO:15, and (iii) CDR-L3 comprising the amino acid sequence of SEQ ID NO:16 wherein the CDRs of the anti-HIV 1 Env CD4bs antibody are defined by the Kabat numbering scheme. See Table 1.

In some embodiments, provided herein is an anti-HIV-I Env antibody or antigen-binding fragment thereof comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 1 (VH of anti-HIV-1 Env V1/V2) or SEQ ID NO:9 (VH of anti-HIV-1 Env CD4bs). In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:1 or SEQ ID NO:9 contains substitutions (e.g., conservative substitutions), insertions, or deletions relative to the reference sequence and retains the ability to bind to HIV-1 Env. In certain embodiments, a total of 1 to 10 amino acids have been substituted, inserted and/or deleted in SEQ ID NO:1 or SEQ ID NO:9. In certain embodiments, substitutions, insertions, or deletions (e.g., 1, 2, 3, 4, or 5 amino acids) occur in regions outside the CDRs (i.e., in the FRs). In some embodiments, the anti-HIV-I Env antibody comprises a heavy chain variable domain sequence of SEQ ID NO:1 or SEQ ID NO:9 including post-translational modifications of that sequence. In certain embodiments, a heavy chain variable domain sequence contains one point mutation relative to SEQ ID NO:1 or SEQ ID NO:9, In further embodiments, the one point mutation is located in a CDR region.

In some embodiments, provided herein is an anti-HIV-1 Env antibody or antigen-binding fragment thereof comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:5 (VL of anti-HIV-1 Env V1/V2) or SEQ ID NO:13 (VL of anti-HIV-1 Env CD4bs). In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:5 or SEQ ID NO:13 contains substitutions (e.g., conservative substitutions), insertions, or deletions relative to the reference sequence and retains the ability to bind to HIV-I Env. In certain embodiments, a total of 1 to 10 amino acids have been substituted, inserted and/or deleted in SEQ ID NO:5 or SEQ ID NO:13. In certain embodiments, substitutions, insertions, or deletions (e.g., 1, 2, 3, 4, or 5 amino acids) occur in regions outside the CDRs (i.e., in the FRs). In some embodiments, the anti-HIV-1 Env antibody comprises a light chain variable domain sequence of SEQ ID NO:5 or SEQ ID NO:13 including post-translational modifications of that sequence. In certain embodiments, a light chain variable domain sequence contains at least one point mutation relative to SEQ ID NO:5 or SEQ ID NO:13. In further embodiments, the one point mutation is located in a CDR region.

In specific embodiments, provided herein is an anti-CD16 antibody or antigen-binding fragment thereof comprising a heavy chain variable domain comprising the amino acid sequence of SEQ ID NO: 17 and comprising a light chain variable domain comprising the amino acid sequence of SEQ ID NO:21. See Table 1.

In particular embodiments, provided herein is an anti-CD16 antibody or antigen-binding fragment thereof comprising a heavy chain variable region and a light chain variable region, wherein the heavy chain variable region comprises (i) CDR-H1 comprising the amino acid sequence of SEQ ID NO:18, (ii) CDR-H2 comprising the amino acid sequence of SEQ ID NO:19, and (iii) CDR-H3 comprising the amino acid sequence of SEQ ID NO:20; and/or wherein the light chain variable region comprises (i) CDR-L1 comprising the amino acid sequence of SEQ ID NO:22, (ii) CDR-L2 comprising the amino acid sequence of SEQ ID NO:23, and (iii) CDR-L3 comprising the amino acid sequence of SEQ ID NO:24 wherein the CDRs of the anti-CD16 antibody are defined by the Kabat numbering scheme. See Table 1.

In some embodiments, provided herein is an anti-CD16 antibody or antigen-binding fragment thereof comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:17, In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:17 contains substitutions (e.g., conservative substitutions), insertions, or deletions relative to the reference sequence and retains the ability to bind to CD16. In certain embodiments, a total of 1 to 10 amino acids have been substituted, inserted and/or deleted in SEQ ID NO:17. In certain embodiments, substitutions, insertions, or deletions (e.g., 1, 2, 3, 4, or 5 amino acids) occur in regions outside the CDRs (i.e., in the FRs). In some embodiments, the anti-CD16 antibody comprises a heavy chain variable domain sequence of SEQ ID NO:17 including post-translational modifications of that sequence. In certain embodiments, a heavy chain variable domain sequence contains one point mutation relative to SEQ ID NO:17. In further embodiments, the one point mutation is located in a CDR region.

In some embodiments, provided herein is an anti-CD16 antibody or antigen-binding fragment thereof comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:21. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:21 contains substitutions (e.g., conservative substitutions), insertions, or deletions relative to the reference sequence and retains the ability to bind to CD16. In certain embodiments, a total of 1 to 10 amino acids have been substituted, inserted and/or deleted in SEQ ID NO:21. In certain embodiments, substitutions, insertions, or deletions (e.g., 1, 2, 3, 4, or 5 amino acids) occur in regions outside the CDRs (i.e., in the FRs). In some embodiments, the anti-CD16 antibody comprises a light chain variable domain sequence of SEQ ID NO:21 including post-translational modifications of that sequence. In certain embodiments, a light chain variable domain sequence contains at least one point mutation relative to SEQ ID NO:21. In further embodiments, the one point mutation is located in a CDR region.

Thus, in particular embodiments, the sequences can comprise at least one conservative substitution. It is understood that the phrase “at least one” is synonymous with “one or more” and includes values such as at least 1, at least 2, at least 3, at least 4, at least 5, at least 6, at least 7, at least 8, at least 9, at least 10, at least 11, at least 12, at least 13, at least 14, at least 15 . . . at least “N” wherein “N” equals the total number of amino acids in the particular sequence (and therefore, 1 or more, 2 or more, 3 or more, etc.).

The sequences can also comprise up to 1, up to 2, up to 3, up to 4, up to 5, up to 6, up to 7, up to 8, up to 9, up to 10, up to 11, up to 12, up to 13, up to 14, up to 15 . . . up to “N” wherein “N” equals the total number of amino acids in the particular sequence. Alternatively, a particular sequence can comprise a substitution at 10 or fewer, 9 or fewer, 8 or fewer, 7 or fewer, 6 or fewer, 5 or fewer, 4 or fewer, 3 or fewer, 2 or fewer, etc., amino acid positions.

In specific embodiments, the present invention provides an isolated antibody or antibody-binding fragment thereof that specifically binds to HIV-1 Env V1/V2, wherein the antibody or antibody-binding fragment comprises heavy chain complementarity determining regions (CDRs) 1, 2 and 3, wherein the heavy chain CDR1 comprises an amino acid sequence as set forth in SEQ ID NO:2, or the amino acid sequence as set forth in SEQ ID NO:2 with a substitution at three or fewer amino acid positions, the heavy chain CDR2 comprising an amino acid set forth in SEQ ID NO:3, or the amino acid set forth in SEQ ID NO:3 with a substitution at seven or fewer amino acid positions, and the heavy chain CDR3 comprising an amino acid sequence as set forth in SEQ ID NO:4, or the amino acid sequence as set forth in SEQ ID NO:4 with a substitution at four or fewer amino acid positions.

In further embodiments, the isolated antibody or antigen-binding fragment further comprises light chain CDRs 1, 2 and 3, wherein the light chain CDR1 comprises an amino acid sequence as set forth in SEQ ID NO:6, or the amino acid sequence as set forth in SEQ ID NO:6 with a substitution at six or fewer amino acid positions, the light chain CDR2 comprising an amino acid sequence as set forth in SEQ ID NO:7, or the amino acid sequence as set forth in SEQ ID NO:7 with a substitution at four or fewer amino acid positions, and the light chain CDR3 comprising an amino acid sequence as set forth in SEQ ID NO:8, or the amino acid sequence as set forth in SEQ ID NO:8 with a substitution at five or fewer amino acid positions.

In specific embodiments, the present invention provides an isolated antibody or antibody-binding fragment thereof that specifically binds to HIV-1 Env CD4bs, wherein the antibody or antibody-binding fragment comprises heavy chain complementarity determining regions (CDRs) 1, 2 and 3, wherein the heavy chain CDR1 comprises an amino acid sequence as set forth in SEQ ID NO:10, or the amino acid sequence as set forth in SEQ ID NO:10 with a substitution at three or fewer amino acid positions, the heavy chain CDR2 comprising an amino acid set forth in SEQ ID NO:11, or the amino acid set forth in SEQ ID NO:11 with a substitution at seven or fewer amino acid positions, and the heavy chain CDR3 comprising an amino acid sequence as set forth in SEQ ID NO:12, or the amino acid sequence as set forth in SEQ ID NO:12 with a substitution at four or fewer amino acid positions.

In further embodiments, the isolated antibody or antigen-binding fragment further comprises light chain CDRs 1, 2 and 3, wherein the light chain CDR1 comprises an amino acid sequence as set forth in SEQ ID NO:14, or the amino acid sequence as set forth in SEQ ID NO:14 with a substitution at six or fewer amino acid positions, the light chain CDR2 comprising an amino acid sequence as set forth in SEQ ID NO:15, or the amino acid sequence as set forth in SEQ ID NO:15 with a substitution at four or fewer amino acid positions, and the light chain CDR3 comprising an amino acid sequence as set forth in SEQ ID NO:16, or the amino acid sequence as set forth in SEQ ID NO:16 with a substitution at five or fewer amino acid positions.

In specific embodiments, the present invention provides an isolated antibody or antibody-binding fragment thereof that specifically binds to CD16, wherein the antibody or antibody-binding fragment comprises heavy chain complementarity determining regions (CDRs) 1, 2 and 3, wherein the heavy chain CDR1 comprises an amino acid sequence as set forth in SEQ ID NO:18, or the amino acid sequence as set forth in SEQ ID NO:18 with a substitution at three or fewer amino acid positions, the heavy chain CDR2 comprising an amino acid set forth in SEQ ID NO:19, or the amino acid set forth in SEQ ID NO:19 with a substitution at seven or fewer amino acid positions, and the heavy chain CDR3 comprising an amino acid sequence as set forth in SEQ ID NO:20, or the amino acid sequence as set forth in SEQ ID NO:20 with a substitution at four or fewer amino acid positions.

In further embodiments, the isolated antibody or antigen-binding fragment further comprises light chain CDRs 1, 2 and 3, wherein the light chain CDR1 comprises an amino acid sequence as set forth in SEQ ID NO:22, or the amino acid sequence as set forth in SEQ ID NO:22 with a substitution at six or fewer amino acid positions, the light chain CDR2 comprising an amino acid sequence as set forth in SEQ ID NO:23, or the amino acid sequence as set forth in SEQ ID NO:23 with a substitution at four or fewer amino acid positions, and the light chain CDR3 comprising an amino acid sequence as set forth in SEQ ID NO:24, or the amino acid sequence as set forth in SEQ ID NO:24 with a substitution at five or fewer amino acid positions.

The antibodies also include derivatives that are modified, i.e., by the covalent attachment of any type of molecule to the antibody such that covalent attachment does not prevent the antibody from binding to HIV-1 Env/CD16 or from exerting a cytostatic or cytotoxic effect on cells. For example, but not by way of limitation, the antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc., Any of numerous chemical modifications may be carried out by known techniques, including, but not limited to specific chemical cleavage, acetylation, formylation, metabolic synthesis of tunicarnycin, etc., Additionally, the derivative may contain one or more non-classical amino acids.

### A. Linker Sequence

In some embodiments, one or more linkers (e.g., flexible linkers) can be introduced into the bispecific antibody construct to provide flexibility at one or more of the junctions between domains, between moieties, between moieties and domains, or at any other junctions where a linker would be beneficial. In some embodiments, the bispecific antibody construct further comprises a linker sequence. In some embodiments, the linker sequence can be a flexible linker sequence. In some embodiments, the linker sequence is a synthetic linker sequence.

In some embodiments, the linker sequence can include a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments, the linker sequence can include a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids in length.

In some embodiments, a linker sequence can be rich in glycine (Gly or G) residues. In some embodiments, the linker sequence can be rich in serine (Ser or S) residues. In some embodiments, the linker sequence can be rich in glycine and serine residues. In some embodiments, the linker sequence has one or more Gly-Gly-Gly-Gly-Ser (GGGGS or G4S) sequences (e.g., 1, 2, 3, 4, 5, 6, 7, 8, 9, or 10 or more GGGGS sequences). In some embodiments, the linker sequence comprises a (G4S)3 or a (G4S)2 linker. In some embodiments, the linker sequence comprises a GGGGSGGGGSGGGGS (SEQ ID NO:27). In some embodiments, the linker sequence comprises a GGGGSGGGGS (SEQ ID NO:28).

### III. Affinity Maturation

In one embodiment, a bispecific molecule comprising an anti-HIV-1 Env antibody or antigen-binding fragment thereof and an anti-CD16 antibody or antigen-binding fragment thereof is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used. Methods of effecting affinity maturation include random mutagenesis (e.g., Fukuda et al., Nucleic Acids Res., 34:e127 (2006); targeted mutagenesis (e.g., Rajpal et al., Proc. Natl. Acad. Sci. USA, 102:8466-71 (2005); shuffling approaches (e.g., Jermutus et al., Proc. Natl. Acad. Sci. USA, 98:75-80 (2001); and in silica approaches (e.g., Lippow et al., Nat. Biotechnol., 25:1171-6 (2005).

In some embodiments, the mutagenesis is targeted to regions known or likely to be at the binding interface. For example, mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In other embodiments, mutagenesis can also be limited to one or a few of the CDRs, e.g., to make step-wise improvements.

In other embodiments, the bispecific molecule comprising an anti-HIV-1 Env antibody or antigen-binding fragment thereof and anti-CD16 antibody or antigen-binding fragment thereof may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half-life by providing a salvage receptor binding epitope.

In one embodiment, a bispecific molecule comprising an anti-HIV-1 Env antibody or antigen-binding fragment thereof and an anti-CD16 antibody or antigen-binding fragment thereof has one or more CDR sequences (e.g., a Chothia, an enhanced Chothia, or Kabat CDR) that differ from those described herein. In one embodiment, the antibody has one or more CDR sequences that include amino acid changes, such as substitutions of 1,2, 3, or 4 amino acids if a CDR is 5-7 amino acids in length, or substitutions of 1, 2, 3, 4, or 5, of amino acids in the sequence of a CDR if a CDR is 8 amino acids or greater in length. The amino acid that is substituted can have similar charge, hydrophobicity, or stereochemical characteristics. In some embodiments, the amino acid substitution(s) is a conservative substitution. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In other embodiments, the amino acid substitution(s) is a non-conservative substitution. The antibody or antibody fragments thereof that contain the substituted CDRs can be screened to identify antibodies of interest.

Unlike in CDRs, more substantial changes in structure framework regions (FRs) can be made without adversely affecting the binding properties of an antibody. Changes to FRs include, but are not limited to, humanizing a non-human-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J Immun., 147:26S7-62 (1991); Morgan et al., Immunology, 86:319-24 (199S)), or changing the species from which the constant region is derived.

### IV. Humanized Antibodies

A humanized antibody is a genetically engineered antibody in which the CDRs from a non-human “donor” antibody are grafted into human “acceptor” antibody sequences. See, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. In one embodiment, an acceptor sequence for the heavy chain is the germline V_{H} exon V_{H}1-2 (also referred to in the literature as HV1-2) (Shin et al, 1991, EMBO J. 10:3641-3645) and for the hinge region (JH), exon JH-6 (Mattila et al, 1995, Eur. J. Immunol. 25:2578-2582). For the light chain, an acceptor sequence can comprise exon VK2-30 (also referred to in the literature as KV2-30) and for the hinge region exon JK-4 (Hieter et al, 1982, J. Biol. Chem. 257:1516-1522). Thus, a humanized antibody is an antibody having some or all CDRs entirely or substantially from a donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 60%, 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical. In some embodiments, the bispecific antibodies of the invention are humanized antibodies.

Although humanized antibodies often incorporate all six CDRs (preferably as defined by Kabat) from a mouse antibody, they can also be made with less than all CDRs (e.g., at least 3, 4, or 5) CDRs from a mouse antibody. See, e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; and Tamura et al, Journal of Immunology, 164:1432-1441, 2000.

### V. Pharmaceutical Compositions

The present disclosure also provides pharmaceutical compositions comprising one or more of: (i) bispecific molecule comprising an anti-HIV-1 Env antibody or antigen-binding fragment thereof and an anti-CD16 antibody or antigen-binding fragment thereof disclosed herein; (ii) a nucleic acid molecule or the set of nucleic acid molecules encoding a bispecific molecule as disclosed herein; or (iii) a vector or set of vectors disclosed herein, and a pharmaceutically acceptable carrier.

A bispecific molecule comprising an anti-HIV-1 Env antibody or antigen-binding fragment thereof and an anti-CD16 antibody or antigen-binding fragment thereof described herein can be formulated as a pharmaceutical composition for administration to a subject, e.g., to treat HIV. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, a bispecific antibody composition described herein is formulated with excipient materials, such as sodium citrate, sodium dibasic phosphate heptahydrate, sodium monobasic phosphate, Tween®-80, and a stabilizer. It can be provided, for example, in a buffered solution at a suitable concentration and can be stored at 2-8° C. In some other embodiments, the pH of the composition is between about 5.5 and 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, and 7.5).

The pharmaceutical compositions can also include agents that reduce aggregation of the antibody when formulated. Examples of aggregation reducing agents include one or more amino acids selected from the group consisting of methionine, arginine, lysine, aspartic acid, glycine, and glutamic acid. These amino acids may be added to the formulation to a concentration of about 0.5 mM to about 145 mM (e.g., 0.5 mM, 1 mM, 2 mM, 5 mM, 10 mM, 25 mM, 50 mM, 100 mM). The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

The bispecific antibody composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization.

Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the bispecific antibody compositions may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In one embodiment, the pharmaceutical formulation comprises a bispecific antibody composition at a concentration of about 0.005 mg/mL to 500 mg/mL (e.g., 0.005 mg/ml, 0.01 mg/ml, 0.05 mg/ml, 0.1 mg/ml, 0.5 mg/mL, 1 mg/mL, 5 mg/mL, 10 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 250 mg/mL, 300 mg/mL, 350 mg/mL, 400 mg/mL, 450 mg/mL, 500 mg/mL), formulated with a pharmaceutically acceptable carrier. In some embodiments, the antibody is formulated in sterile distilled water or phosphate buffered saline. The pH of the pharmaceutical formulation may be between 5.5 and 7.5 (e.g., 5.5, 5.6, 5.7, 5.8, 5.9, 6.0, 6.1, 6.2 6.3, 6.4 6.5, 6.6 6.7, 6.8, 6.9 7.0, 7.1, 7.3, 7.4, 7.5).

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

The antibodies or antigen-binding fragment thereof, or nucleic acids encoding same of the disclosure can be administered to a subject, e.g., a subject in need thereof, for example, a human or animal subject, by a variety of methods. For many applications, the route of administration is one of: intravenous injection or parenteral, infusion (IV), subcutaneous injection (SC), intraperitoneally (IP), or intramuscular injection, intratumor (IT). Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

In one embodiment, the route of administration of the bispecific antibody compositions of the present invention is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the invention, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g., polysorbate), optionally a stabilizer agent (e.g., human albumin), etc., However, in other methods compatible with the teachings herein, the bispecific antibody composition can be delivered directly to a particular site.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a bispecific antibody composition by itself or in combination with other active agents) in the required amount in an appropriate solvent with one or a combination of ingredients enumerated herein, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit.

The bispecific antibody composition can be administered as a fixed dose, or in a mg/kg dose. The dose can also be chosen to reduce or avoid production of antibodies against the bispecific antibody composition. Dosage regimens are adjusted to provide the desired response, e.g., a therapeutic response or a combinatorial therapeutic effect. Generally, doses of the bispecific antibody composition (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In certain embodiments, a subject in need of treatment with a bispecific antibody composition is administered the antibody or fragment thereof at a dose of between about 1 mg/kg to about 30 mg/kg. In some embodiments, a subject in need of treatment with a bispecific antibody composition is administered the composition at a dose of 1 mg/kg, 2 mg/kg, 4 mg/kg, 5 mg/kg, 7 mg/kg 10 mg/kg, 12 mg/kg, 15 mg/kg, 20 mg/kg, 25 mg/kg, 28 mg/kg, 30 mg/kg, 35 mg/kg, 40 mg/kg, or 50 mg/kg. In a specific embodiment, the bispecific antibody composition is administered subcutaneously at a dose of 1 mg/kg to 3 mg/kg. In another embodiment, the composition is administered intravenously at a dose of between 4 mg/kg and 30 mg/kg.

A bispecific antibody composition may comprise about 1 mg/mL to 100 mg/ml or about 10 mg/mL to 100 mg/ml or about 50 to 250 mg/mL or about 100 to 150 mg/ml or about 100 to 250 mg/ml.

Dosage unit form or “fixed dose” as used herein refers to physically discrete units suited as unitary dosages for the subjects to be treated; each unit contains a predetermined quantity of the bispecific antibody composition calculated to produce the desired therapeutic effect in association with the required pharmaceutical carrier and optionally in association with the other agent. Single or multiple dosages may be given. Alternatively, or in addition, the composition may be administered via continuous infusion.

A bispecific antibody composition dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of a bispecific antibody composition can include a single treatment or, preferably, can include a series of treatments.

Ifa subject is at risk for developing a disorder described herein, the bispecific antibody composition can be administered before the full onset of the disorder, e.g., as a preventative measure. The duration of such preventative treatment can be a single dosage of the bispecific antibody composition or the treatment may continue (e.g., multiple dosages). For example, a subject at risk for the disorder may be treated with the bispecific antibody composition for days, weeks, months, or even years so as to prevent the disorder from occurring or fulminating.

In certain embodiments, the bispecific antibody composition is administered subcutaneously at a concentration of about 1 mg/mL to about 500 mg/mL (e.g., 1 mg/mL, 2 mg/mL, 3 mg/mL, 4 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 400 mg/mL, 450 mg/mL). In one embodiment, the bispecific antibody composition is administered subcutaneously at a concentration of 50 mg/mL. In another embodiment, the bispecific antibody composition is administered intravenously at a concentration of about 1 mg/mL to about 500 mg/mL. In one embodiment, the bispecific antibody composition is administered intravenously at a concentration of 50 mg/mL.

Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months.

Bispecific antibody compositions of the present invention can be administered on multiple occasions. Intervals between single dosages can be daily, weekly, monthly or yearly. Intervals can also be irregular as indicated by measuring blood levels of, for example, HIV-1 Env antigen in the patient. Alternatively, bispecific antibody compositions can be administered as a sustained release formulation, in which case less frequent administration is required. Dosage and frequency vary depending on the half-life of the composition in the patient.

The dosage and frequency of administration can vary depending on whether the treatment is prophylactic or therapeutic. In prophylactic applications, compositions containing the polypeptides of the invention or a cocktail thereof are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

### VI. Combination Therapy

The bispecific antibodies of the present invention can be administered in combination with an HIV-1 treatment including, but not limited to antiretroviral therapy (ART) and latency reversing agents (LRA). One example of an LRA is bisphosphonate. In a specific embodiment, the bisphosphonate may be an aminobisphosphonate. The aminobisphosphonate may be an alkyl aminobisphosphonate, a substituted alkyl aminobisphosphonate, a bisphosphonate with a nitrogen containing heterocycle, or a bisphosphonate containing a cyclic alkane or cycloaminoalkane. In some embodiments, the aminobisphosphonate is Alendronate (Fosamax™), Ibandronate (Boniva™ or Bonviva™) Neridronate (Nerixia™), Olpadronate, Pamidronate (APD/Aredia™), Risedronate (Actonel™), or Zoledronate (Zometa™/Aclasta™).

Other examples of LRAs include, but are not limited to, an epigenetic modifier, an NFkB agonist, a PI3K Akt pathway inhibitor, a protein kinase C agonist, a TCR activator, a TLR agonist, a second mitochondrial-derived activator of caspases (SMAC) mimetic, an inhibitor of IAP (Inhibitor of Apoptosis Protein) family of proteins, or a stimulator of interferon genes protein (STING) agonists.

An example of an epigenetic modifier LRA is a histone deacetylase (HDAC) inhibitor. The HDAC inhibitor may be belinostat (PXD101), entinostat (MS-275), mocetinostat (MGCD0103), panobinostat (LBH589), romidepsin or vorinostat (SAHA), trapoxin, trichostatin A, or valproic acid.

Another example of an LRA is BAF complex modulating compound (see claims 1-26 of International Application Number PCT/US2019/041466, filed Jul. 11, 2019 (The Board of Trustees of the Leland Stanford Junior University).

In other embodiments, an LRA comprises histone deacetylase inhibitors (Trichostatin A, trapoxin, suberoylanilide hydroxamic acid, romidepsin, vorinostat, panobinostat, entinostat, valproic acid, fimepinostat, and chidamide); histone methyltransferase inhibitors (Chaetocin and BIX-01294); DNA methylation inhibitors (5-aza-cytidine, 5-aza-deoxycytidine, and zebularine); PKC agonists (Phorbol 12-myristate 13-acetate, prostratin, SUW013 and C13 analogs, bryostatin-1, and ingenol and analogs); BET inhibitors (JQ1, I-BET, I-BET151, MMQO, RVX-208, and PFI-1); TLR agonists (Flagellin, Pam3 CSK4, GS-9620, and MGN1703); activators of Akt pathway (Disulfiram and hexamethylene bisacetamide); SMAC mimetics (SBI-0637142, SBI-0953294, and AZD5582); STAT5 sumoylation inhibitors (1-hydroxybenzotriazol, 1-hydroxy-7-amino benzotriazole, and 3-hydroxy-1,2,3-benzotriazin-4(3H)-one and analogs) and prodrugs LRAs (Prodrugs of PKC agonists).

### VII. Devices and Kits for Therapy

An anti-HIV-1 Env/anti-CD16 bispecific antibody or antigen-binding fragment thereof can be provided in a kit. In one embodiment, the kit includes (a) a container that contains a composition that includes an anti-HIV-1 Env/anti-CD16 bispecific antibody or antigen-binding fragment thereof as described herein, and optionally (b) informational material. The informational material can be descriptive, instructional, marketing or other material that relates to the methods described herein and/or the use of the agents for therapeutic benefit.

In certain embodiments, the kit also includes a second agent for treating HIV (e.g., latency reversing agent). For example, the kit includes a first container that contains a composition that includes the anti-HIV-1 Env/anti-CD16 bispecific antibody or antigen-binding fragment thereof, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-HIV-1 Env/anti-CD16 bispecific antibody or antigen-binding fragment thereof, e.g., in a suitable dose, dosage form, or mode of administration (e.g., a dose, dosage form, or mode of administration described herein), to treat a subject who has had or who is at risk for a disease as described herein. The information can be provided in a variety of formats, include printed text, computer readable material, video recording, or audio recording, or information that provides a link or address to substantive material, e.g., on the internet.

In addition to the anti-HIV-1 Env/anti-CD16 bispecific antibody or antigen-binding fragment thereof, the composition in the kit can include other ingredients, such as a solvent or buffer, a stabilizer, or a preservative. The anti-HIV-1 Env/anti-CD16 bispecific antibody or antigen-binding fragment thereof can be provided in any form, e.g., liquid, dried or lyophilized form, preferably substantially pure and/or sterile. When the agents are provided in a liquid solution, the liquid solution preferably is an aqueous solution. In certain embodiments, the anti-HIV-1 Env/anti-CD16 bispecific antibody or antigen-binding fragment thereof in the liquid solution is at a concentration of about 25 mg/mL to about 250 mg/mL (e.g., 40 mg/mL, 50 mg/mL, 60 mg/mL, 75 mg/mL, 85 mg/mL, 100 mg/mL, 125 mg/mL, 150 mg/mL, and 200 mg/mL). When the anti-HIV-1 Env/anti-CD16 bispecific antibody or antigen-binding fragment thereof is provided as a lyophilized product, the anti-HIV-1 Env/anti-CD16 bispecific antibody or antigen-binding fragment thereof is at about 75 mg/vial to about 200 mg/vial (e.g., 100 mg/vial, 108.5 mg/vial, 125 mg/vial, 150 mg/vial). The lyophilized powder is generally reconstituted by the addition of a suitable solvent. The solvent, e.g., sterile water or buffer (e.g., PBS), can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-HIV-1 Env/anti-CD16 bispecific antibody or antigen-binding fragment thereof and the second agent, e.g., a latency reversing agent, in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Bispecific Antibodies Promote Natural Killer Cell-Mediated Elimination of HIV-1 Reservoir Cells

The persistence of CD4+ T cells carrying latent human immunodeficiency virus-1 (HIV-1) proviruses is the main barrier to a cure. New therapeutics to enhance HIV-1-specific immune responses and clear infected cells will probably be necessary to achieve reduction of the latent reservoir. In the present study, we report two single-chain diabodies (scDbs) that target the HIV-1 envelope protein (Env) and the human type III Fc receptor (CD16). We show that the scDbs promoted robust and HIV-1-specific natural killer (NK) cell activation and NK cell-mediated lysis of infected cells. Cocultures of CD4+ T cells from people with HIV-1 on antiretroviral therapy (ART) with autologous NK cells and the scDbs resulted in marked elimination of reservoir cells that was dependent on latency reversal. Treatment of human interleukin-15 transgenic NSG mice with one of the scDbs after ART initiation enhanced NK cell activity and reduced reservoir size. Thus, HIV-1-specific scDbs merit further evaluation as potential therapeutics for clearance of the latent reservoir.

### Materials and Methods

Human samples. Leukapheresis samples from people on suppressive ART were obtained from the University of Pennsylvania (BEAT-HIV Delaney cohort, DEL-SPC) and the University of California San Francisco (SCOPE cohort). Selection criteria included undetectable plasma HIV-1 RNA levels (<50 copies per ml) for >72 months. In total, samples from 11 male HIV+ participants were collected (median age=56 years, range=34-69 years; median time on ART=289 months, range=130-360 months; median time on suppressive ART=185 months, range=79-275 months; see Table 2 for ART regimens). This protocol was approved by the institutional review boards at the University of Pennsylvania and the University of California San Francisco. All participants provided written informed consent. Leukapheresis samples from three HIV-participants (two women and one man, aged 29, 46 and 48 years) were obtained from Stemcell Technologies. PBMCs were purified from leukapheresis samples by density gradient centrifugation with Ficoll Paque Plus (GE Healthcare) and cryopreserved.

ScDb production. The gBlocks (IDT) encoding the scDb, an amino-terminal IL-2 signal sequence and a carboxy-terminal 6×His tag were cloned into the pcDNA3.4 vector (Thermo Fisher Scientific). ScDbs were then transiently expressed in Expi293 cells by GeneArt (Thermo Fisher Scientific) and purified from culture supernatant by HisTrap column (GE Healthcare), followed by size exclusion chromatography with a HiLoad Superdex 200 16/600 column (GE Healthcare). Analytical chromatography was performed using a TSKgel G3000SWxl column (TOSOH Bioscience) and a running buffer of 50 mM sodium phosphate and 300 mM sodium chloride, pH 7, at a flow rate of 1.0 ml min-1.

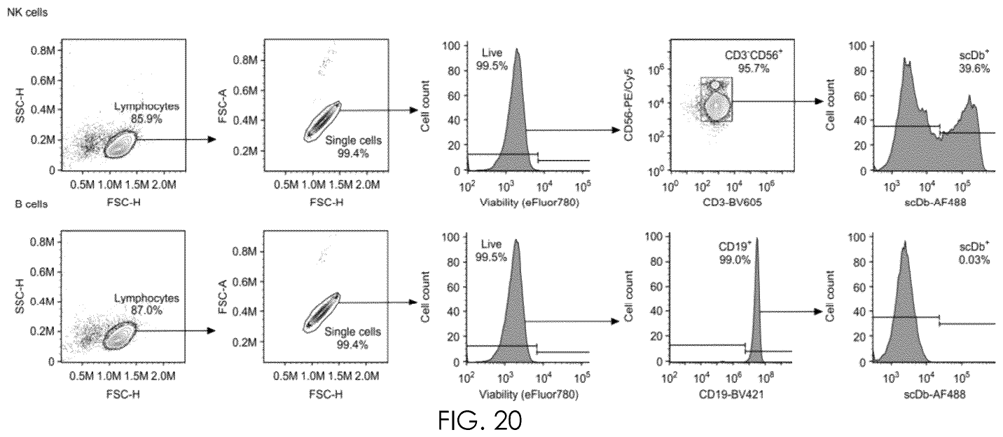

NK cell-surface CD16-binding assay. NK and B cells were isolated from uninfected participant PBMCs (Stemcell Technologies), plated in BCM (RPMI 1640 with 10% fetal bovine serum (FBS) and 1% penicillin-streptomycin) with or without CD16 mIgG-blocking antibody (BioLegend, catalog no. 302001, 1 g ml-1), and incubated at 37° C. for 1 h. The cells were washed in phosphate-buffered saline (PBS) and stained with scDbs (at indicated concentrations, in PBS) at 4° C. for 1 h. The cells were washed and stained with the following antibodies (1:50 dilution)/dye (1:1,000 dilution) at 4° C. for 45 min: CD3-BV605, CD19-BV421, CD56-PE/Cy5 (BioLegend, catalog nos. 317321, 302233 and 304607, respectively), 6×His tag-AF488 and Fixable Viability Dye-eFluor780 (Thermo Fisher Scientific, catalog nos, MA1-135-A488 and 65-0865-14, respectively). The cells were washed, and samples were acquired on an Intellicyt iQue Screener Plus (Sartorius). Data were analyzed with FlowJo (BD Bioscience; see FIG. 20 for gating example). NK cells were defined as live CD3-CD19-CD56+ lymphocytes, T cells as live CD3+ lymphocytes and B cells as live CD19+ lymphocytes. All conditions were tested in triplicate, mean and s.d. shown.

CD16-binding ELISA. CD16-biotin recombinant proteins (F/V158, Acrobiosystems, 0.5 g ml-1) in BAE blocking buffer (PBS, 0.5% bovine serum albumin, 0.1% sodium azide) were added to an EvenCoat streptavidin-coated plate (R&D Systems) and incubated at 4° C. for 16 h. The plate was washed with 1×TBS-T (1× tris-buffered saline (TBS)+0.05% Tween-20) using a BioTek 405 plate washer and scDbs (at indicated concentrations, in BAE) were plated and incubated at 4° C. for 16 h. The plate was washed as before and HRP (horse radish peroxidase) Protein L secondary (Thermo Fisher Scientific, catalog no. 32420, 0.5 g ml-1 in BAE) was plated and incubated at room temperature for 1 h. The plate was washed and developed in TMB (3,3′,5,5′-tetramethylbenzidine) for 5 min at room temperature before stopping in 1 N sulfuric acid. The optical density at 450 nm (OD450) was read; all conditions were tested in triplicate, mean and s.d. shown.

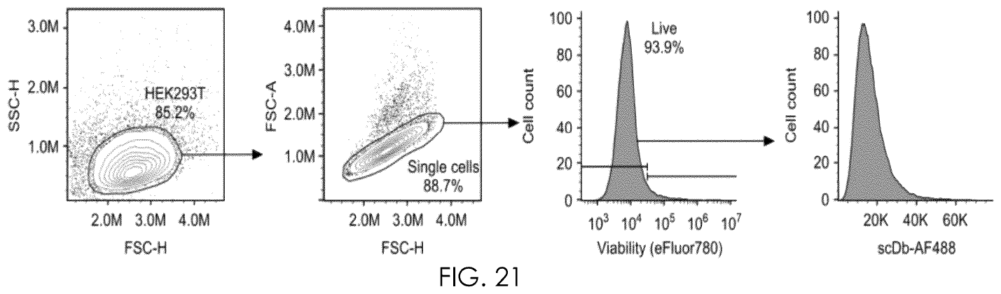

Surface Env-binding assay. HEK293T cells (transformed embryonic kidney cells, American Type Culture Collection, catalog no. CRL-3216) were transfected with a panel of HIV-1 env expression plasmids. Then, 3 d posttransfection, transfected HEK293T cells were stained with scDbs (at indicated concentrations, in Dulbecco's modified Eagle's medium (DMEM) with 10% FBS and 1% penicillin-streptomycin) at 37° C. for 1 h. The cells were washed in PBS and stained with the following secondary antibody/dye at 4° C. for 45 min: 6×His tag-AF488 and Fixable Viability Dye-eFluor780. The cells were washed, and samples were acquired on an Intellicyt iQue Screener Plus. Data were analyzed with FlowJo (see FIG. 21 for gating example). In addition, a subset of transfected HEK293T cells were stained with the parental IgG molecules or an IgG1 isotype control (at 20 g ml-1 in DMEM with 10% FBS and 1% penicillin-streptomycin) to allow for comparison of Env expression. The cells were washed in PBS and stained with the following secondary antibodies (1:50 dilution)/dye (1:1,000 dilution) at 4° C. for 45 min: IgG Fc-BV421 (BioLegend; catalog no. 409318) and Fixable Viability Dye-eFluor780. The cells were washed and samples were acquired on an Intellicyt iQue Screener Plus. Data were analyzed with FlowJo. All conditions were tested in triplicate, mean and s.d. shown.

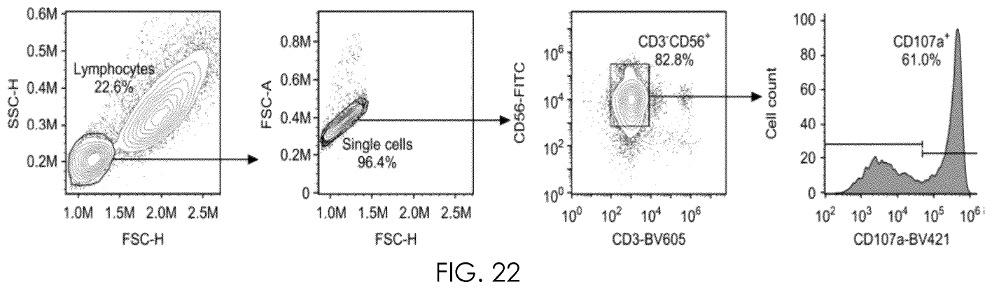

NK activation coculture. A3.01 and ACH-2 cells (transformed CD4+ T cells, National Institutes of Health (NIH) AIDS Reagents Program, catalog nos. ARP-166 and ARP-349, respectively) were plated in BCM with 10 ng ml-1 of TNF and incubated at 37° C. for 18 h. The cells were washed in BCM to remove TNF. NK cells were isolated from uninfected participant PBMC and cocultured with either the reactivated A3.01 or ACH-2 cells at a 1:3 E:T ratio in BCM at 37° C. for 6 h in the presence of the scDbs or the parental IgG molecules (at the indicated concentrations), monensin (BD Bioscience) and CD107a-BV421 (BioLegend, catalog no. 328625). The supernatants were collected and analyzed by Human CD8/NK Panel Legendplex (BioLegend) per the manufacturer's protocol. The cells were washed in PBS and stained with the following antibodies (1:50 dilution)/dye (1:1,000 dilution) at 4° C. for 45 min: CD3-BV605 and CD56-FITC (BioLegend; catalog nos. 317321 and 304603, respectively) and Fixable Viability Dye-eFluor780. The cells were washed, and samples were acquired on an Intellicyt iQue Screener Plus. Data were analyzed with FlowJo (see FIG. 22 for gating example). NK cells are defined as live CD3-CD56+ lymphocytes. All conditions were tested in triplicate, mean and s.d. shown.

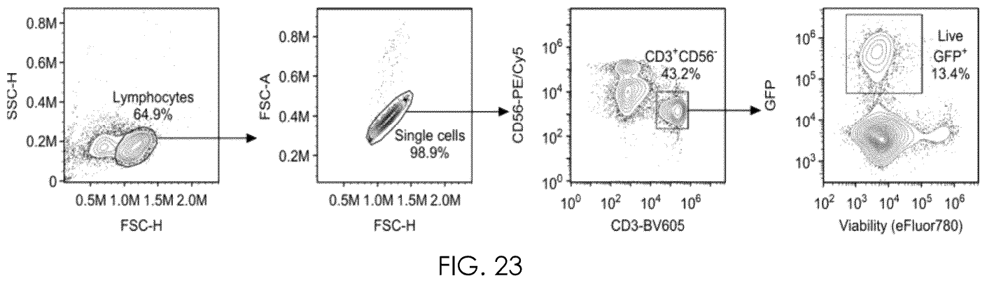

In vitro infected cell elimination coculture. CD4+ T cells were isolated from uninfected participant PBMCs, plated in BCM with IL-2 (30 U ml-1) and CD3/CD28 Dynabeads (Thermo Fisher Scientific) and incubated at 37° C. for 3 d. The activated CD4+T cells were spinoculated with replication-competent NL4.3-ΔNef-eGFP at 100 ng of p24 per 100,000 cells (in conditioned antiretrovirals (ARVs; 10 M tenofovir disoproxil fumarate, 10 μM emtricitabine and 10 M dolutegravir)) were added to the infected cells for 12 h to stop ongoing infection. NK cells were isolated from autologous PBMCs and cocultured with the infected CD4s at a 1:3 E:T ratio in BCM with ARVs at 37° C. for 18 h in the presence of the scDbs or their parental IgG molecules (at indicated concentrations). The cells were washed in PBS and stained with the following antibodies (1:50 dilution)/dye (1:1,000) at 4° C. for 45 min: CD3-BV421, CD56-PE/Cy5 (BioLegend; catalog nos. 317343 and 304607, respectively) and Fixable Viability Dye-eFluor780. The cells were washed and samples were acquired on an Intellicyt iQue Screener Plus. Data were analyzed with FlowJo (see FIG. 23 for gating example). CD4+ T cells were defined as live CD3+CD56− lymphocytes. Supernatant p24 measurements were performed using the Alliance HIV-1 p24 Antigen ELISA kit (PerkinElmer) according to the manufacturer's protocol. Spinoculation of activated CD4+ T cells with replication-competent NL4.3 and subsequent coculture with autologous NK cells and staining were performed as above, except after the initial surface staining; the cells were permeabilized using the Fixation/Permeabilization Kit (BD Bioscience) and stained with p24-FITC (Abcam, catalog no. ab20569, 1:50 dilution) at 4° C. for 45 min before the final wash in PBS and acquisition. Before coculture, a subset of NL4.3-ΔNef-eGFP-infected CD4+ T cells was stained with the parental IgG molecules or an IgG1 isotype control (at 20 g ml-1 in conditioned T cell medium) to measure Env expression. After a 1-h incubation at 37° C., the cells were washed in PBS and stained with the following antibodies (1:50 dilution)/dye (1:1,000) at 4° C. for 45 min: CD3-BV605, human IgG Fc-BV421 (Bio-Legend; catalog nos. 317321 and 409318, respectively) and Fixable Viability Dye-eFluor780. The cells were washed and samples were acquired on an Intellicyt iQue Screener Plus. Data were analyzed with FlowJo. CD4+ T cells were defined as live CD3+ lymphocytes. All conditions were tested in triplicate, mean and s.d. shown.

Ex vivo elimination of latency reversed cell coculture. CD4+ T cells were isolated from PBMCs of participants on suppressive ART (Table 2) and plated in BCM with a stimulating agent and ARVs (10 M tenofovir disoproxil fumarate, 10 M emtricitabine and 10 M dolutegravir) and incubated at 37° C. for 18 h. Stimulating agents were added as follows: bryostatin (10 nM), AZD5582 (500 nM), SAHA (335 nM), romidepsin (40 nM), CD3/CD28 (Dynabeads, Thermo Fisher Scientific, according to the manufacturer's protocol), PMA (50 ng ml-1) and ionomycin (1 μM). The CD4+ T cells were washed in BCM with ARVs to remove the PMA and ionomycin. NK cells were isolated from autologous PBMCs and cocultured with the infected CD4s at a 1:3 E:T ratio in BCM with ARVs at 37° C. for 18 h in the presence of the scDbs (200 pM). CD4+ T cells were re-isolated from the cocultures and depleted of apoptotic cells by annexin-V immunomagnetic selection. DNA isolation and quantification of HIV-1 DNA by IPDA and RPP30 cell equivalents were performed as previously describeds^{51,52}. IPDA measurements were performed in replicates of eight, individual values, mean and s.d. shown. Viable CD4+ T cells were plated at a limiting dilution for QVOAs, which were performed as previously described⁵³, no supernatants were harvested for p24 measurement at day 14. Before coculture, NK cells from a subset of study participants were stained to evaluate surface expression of CD16, NKG2D, Siglec-7, CD57 and PD-1. The cells were incubated with the following antibodies (1:50 dilution)/dye (1:1,000) at 4° C. for 45 min in PBS: CD3-BV605 and CD56-FITC, Fixable Viability Dye-eFluor780 and CD16-BV421, NKG2D-BV421, Siglec-7-PerCP/Cy5.5, CD57-PerCP/Cy5.5 or PD-1-PE/Cy5 (BioLegend; catalog nos. 302038, 320822, 339216, 359622 or 329971, respectively). The cells were washed and samples were acquired on an Intellicyt iQue Screener Plus. Data were analyzed with FlowJo. NK cells were defined as live CD3-CD56+ lymphocytes. All conditions were tested in triplicate, mean and s.d. shown.

Cell-associated polyadenylated HIV-1 RNA measurement. Isolation of caRNA was performed as previously described^{54,55}. Oligo(dT) complementary DNA synthesis was performed using Superscript III Reverse Transcriptase (Thermo Fisher Scientific) previously published methods^{54,55}. All conditions were tested in triplicate, mean and s.d. shown.

Generation of the hIL-15TgNSG mice. The hIL-15TgNSG mice were generated as previously described⁵⁶⁻⁵⁸. All mice experiments were approved by the Wistar Institute Animal Care and Research Committee (protocol no. 201360). All animals recruited in the present study were housed in the Wistar Institute humanized mice holding room with a 12-h light:dark cycle at temperatures of 20-23° C. and 40-60% humidity. Briefly, 6- to 8-week-old female NSG-Tg(hIL-15) (NOD.Cg-Prkdcscid Il2rgtm1Wjl Tg(IL15)1Sz/SzJ; Jackson Laboratory⁵⁹) mice were pretreated with busulfan at 30 mg kg-1 and were then implanted with human fetal thymic tissue fragments and fetal liver tissue fragments under the murine renal capsule. After the surgery, mice were injected via the tail vein with CD34+ hematopoietic stem cells isolated from human fetal liver tissues. Human fetal liver and thymus tissues were procured from Advanced Bioscience Resources. Then 12 weeks postsurgery, human immune cell reconstitution in peripheral blood was determined using a FACSymphony flow cytometer (BD Biosciences) using the following antibodies (1:50 dilution): mCD45-AF700, hCD45-FITC, hCD3-BUV805, hCD4-BUV395, hCD8-PerCP-Cy5.5, hCD56-BV650 and Fixable Viability Stain 510 (catalog nos. 560510, 555482, 612895, 563550, 565310, 564057 and 564406, respectively; BD Biosciences). Data were analyzed with FlowJo.

HIV-1 infection, ART suppression and scDb administration of the hIL-15TgNSG mice. The hIL-15Tg NSG mice were randomly divided into two groups (n=9) and were infected intravenously with 1×10⁴ TCID50 (50% tissue culture infectious dose) of T/F virus HIVSUMA. Peripheral blood was collected weekly for plasma viral load assays. Then 2 weeks post-infection, the mice were placed on a diet combined with ART until the end of the study (1,500 mg kg-1 of emtricitabine, 1,560 mg kg-1 of tenofovir disoproxil fumarate and 600 mg kg-1 of raltegravir). Beginning on day 33 and continuing through day 43 post-infection, the mice were given daily intraperitoneal injections of 200 g (approximately 7 mg kg-1) of either H2-Db or 3BNC117-Db. The mice were euthanized at 7 weeks post-infection and blood and tissues were collected.

Plasma viral load measurement. Plasma viral loads were measured as previously described^{56-58-60,61}. Briefly, viral RNA was extracted using a QIAamp Viral RNA Mini kit (QIAGEN). The pVLs were determined using reverse transcription quantitative PCR (RT-qPCR) on a C1000 Thermal Cycler and the CFX96 Real-Time system (BioRad) with the TaqMan Fast Virus 1-Step Master Mix (Life Technologies).

Measuring markers of T cell and NK cell activation and maturation. Peripheral blood and spleen cell suspensions were collected weekly or at the end of study, respectively, for flow cytometry analysis. Cells were stained using the following antibodies (1:50 dilution): mCD45-AF700, hCD45-FITC, hCD3-BUV805, hCD4-BUV395, hCD8-PerCP-Cy5.5, hCD56-BV650, HLA-DR-APC, hCD38-PE, hCD57-PE-CF594 and Fixable Viability Stain 510 (catalog nos. 560510, 555482, 612895, 563550, 565310, 564057, 340691, 555460, 562488 and 564406, respectively; BD Biosciences). Data were collected on a FACSymphony flow cytometer (BD Biosciences).

The IPDA in hIL-15TgNSG mice. A single-cell suspension of splenocytes was generated using the gentleMACS Octo Dissociator (Miltenyi Biotec). Genomic DNA was extracted from the splenocytes of the IL-15 Tg BLT mice. Each IPDA ddPCR reaction was performed in parallel with a copy reference/shearing correction (RPP30 gene) ddPCR reaction which quantifies input human cell equivalents and DNA shearing⁵¹. Input human CD4+ T cell equivalents were calculated based on flow cytometry staining of the splenocyte samples as follows: (Input human RPP30 cell equivalents)×(percentage hCD4+/hCD45+cells). Shearing corrections were applied to the IPDA data and the final results are reported as copies per 10⁶ human CD4+ T cells.

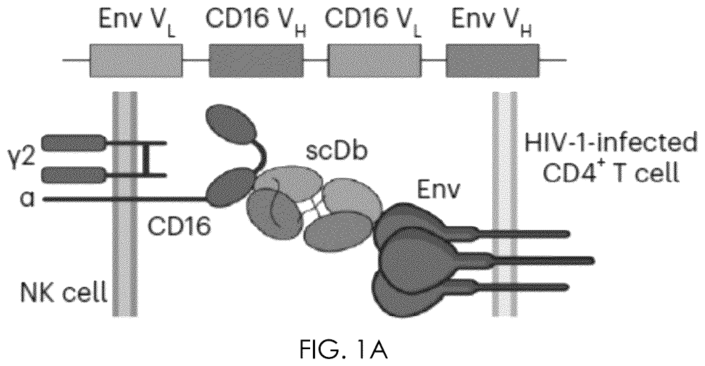

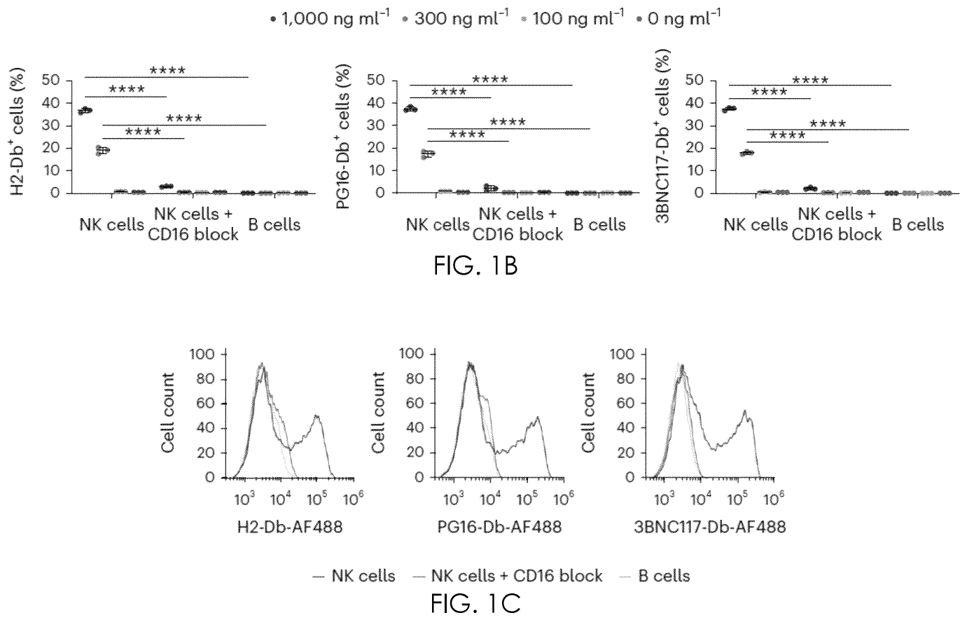

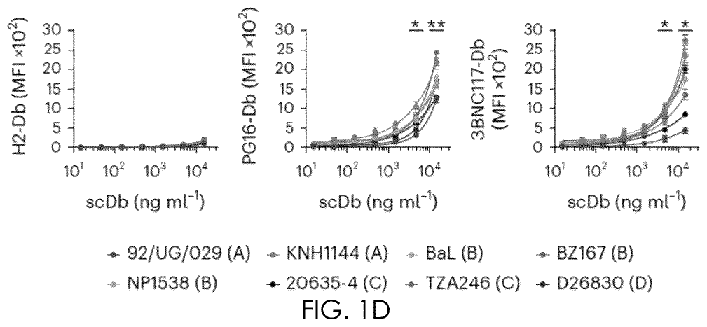

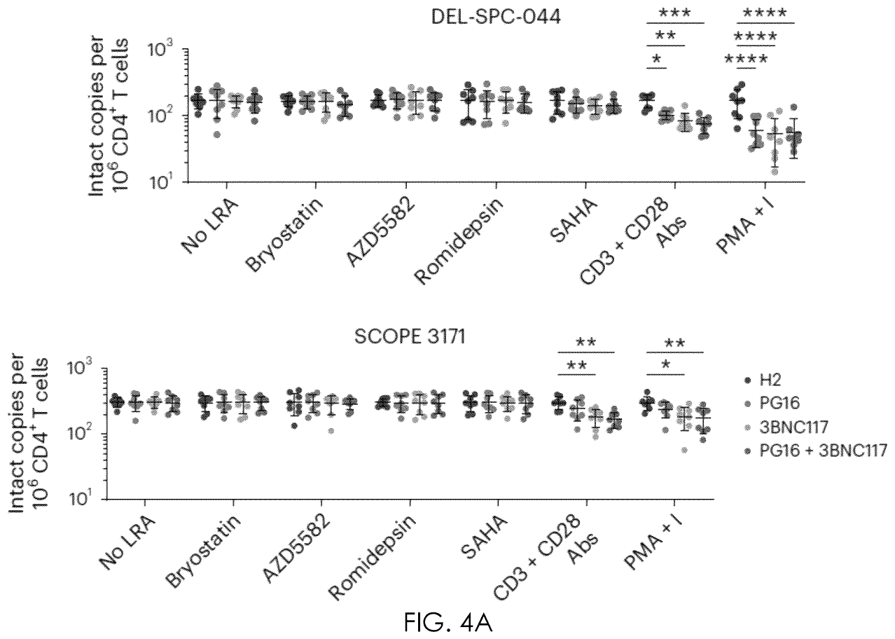

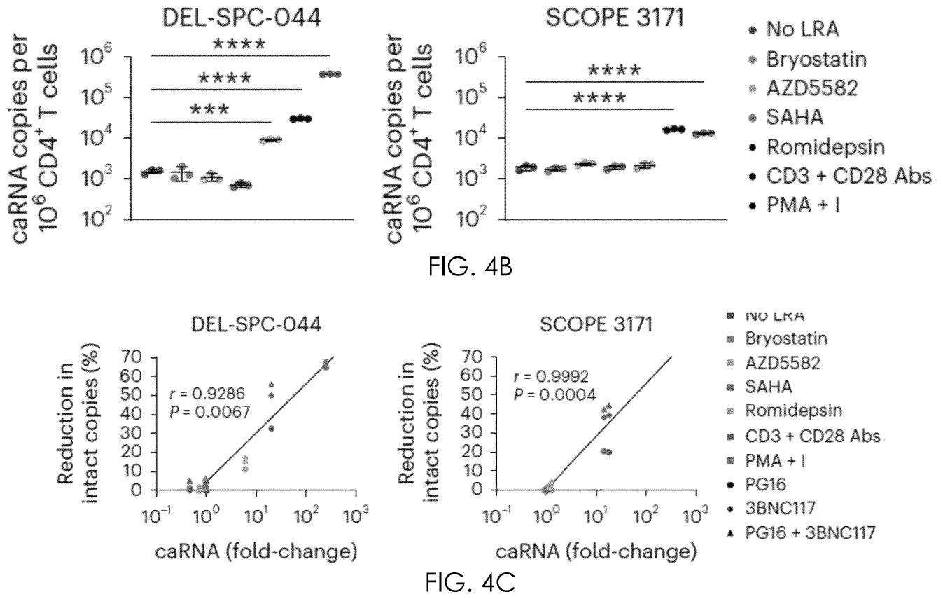

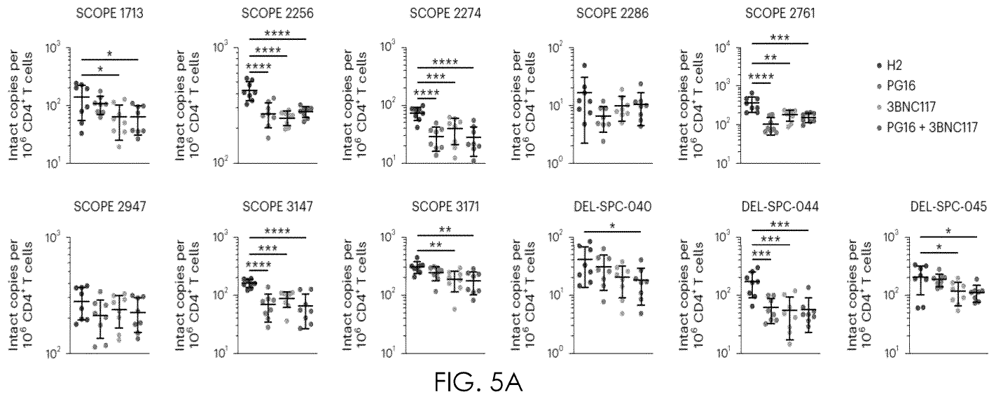

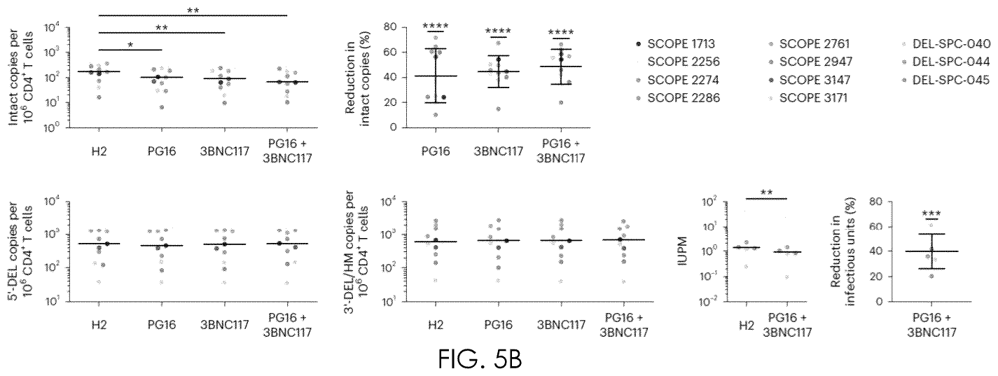

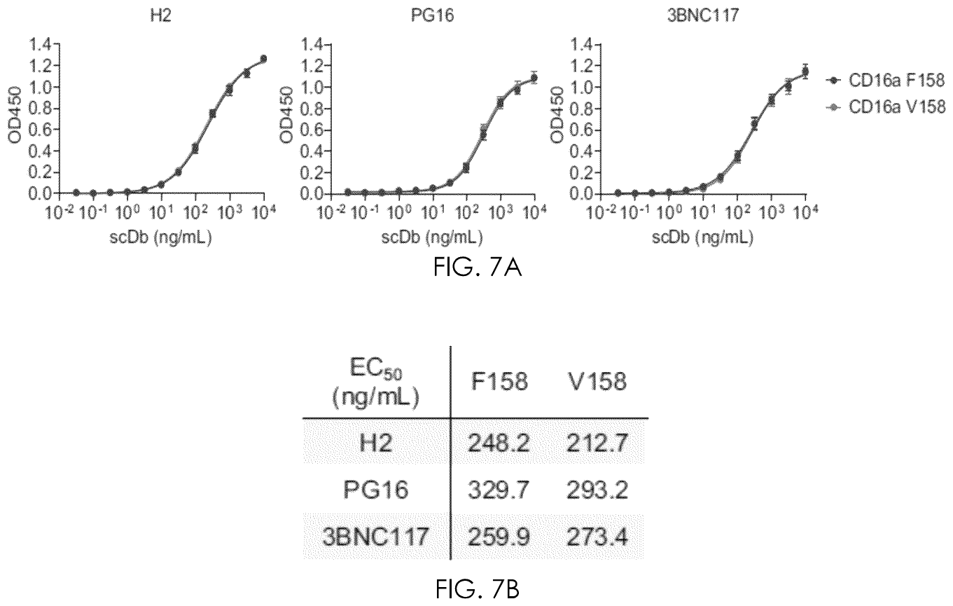

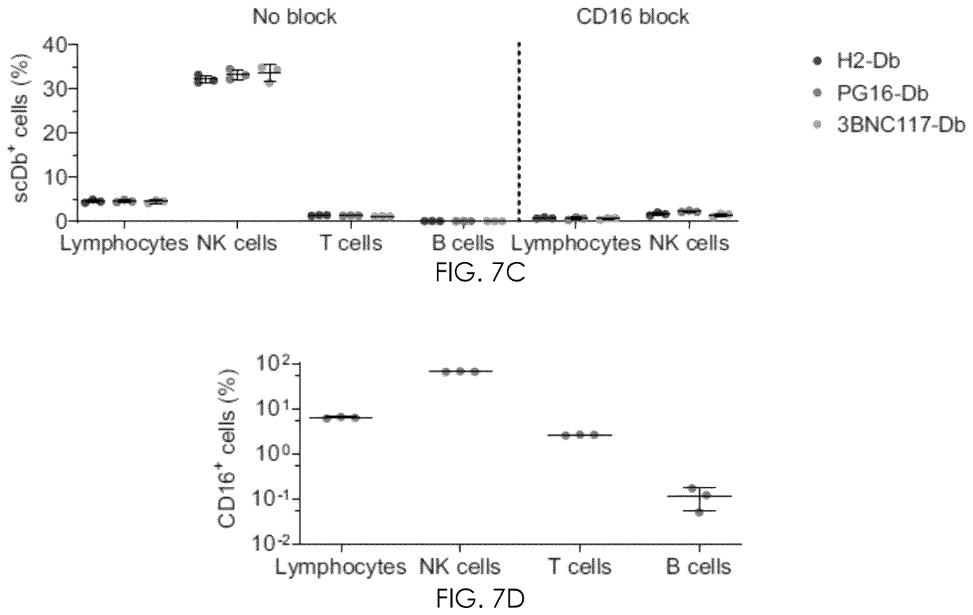

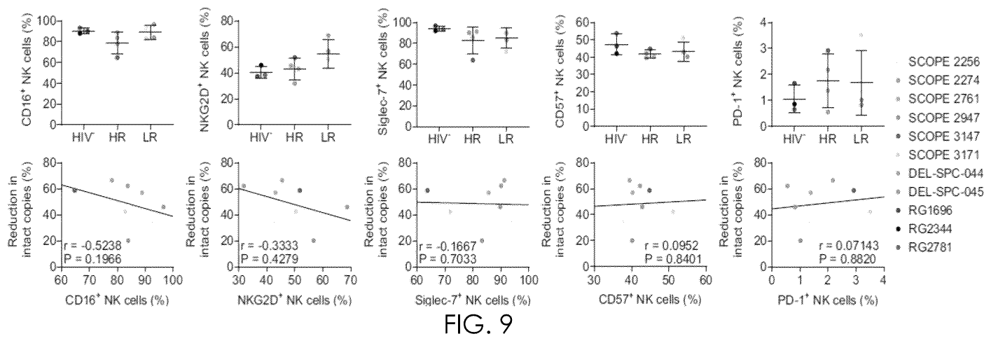

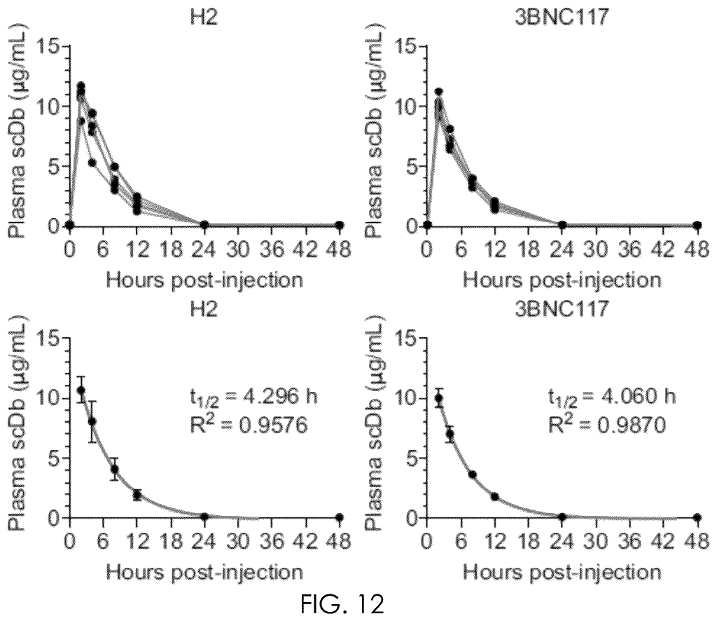

Statistics and reproducibility. Sample sizes are noted for each experiment in the relevant figure legends. No statistical method was used to predetermine sample sizes; they were determined based on availability of biological samples. Controls were included as appropriate to provide a reference with which to compare experimental data. No data were excluded from any analyses. For all in vitro and ex vivo culture experiments, all conditions were tested for each participant in parallel. Assignment of hIL-15TgNSG mice to treatment groups was randomized. During data collection and analysis for all in vitro and ex vivo culture experiments and for hIL-15TgNSG mice-related experiments, the investigators were blinded to the treatment conditions of the relevant biological samples. Statistical tests used in the present study included analysis of variance (ANOVA) with multiple comparisons (FIGS. 1B, 2A, 3B, 4A, 4B, 5A, 5B, 6C, 6D, 8C, 8D and 13-15 and FIG. 19), Student's t-tests (FIGS. 1D and 5B), Spearman's correlations (FIG. 4C and FIG. 9) and nonlinear regression models (FIGS. 7A and 12). All statistical analyses were performed using GraphPad Prism. Data distribution was assumed to be normal but this was not formally tested. P<0.05 was considered significant for all tests. For all figures, *P<0.05, **P<0.01, ***P<0.001 and ****P<0.0001

### Results

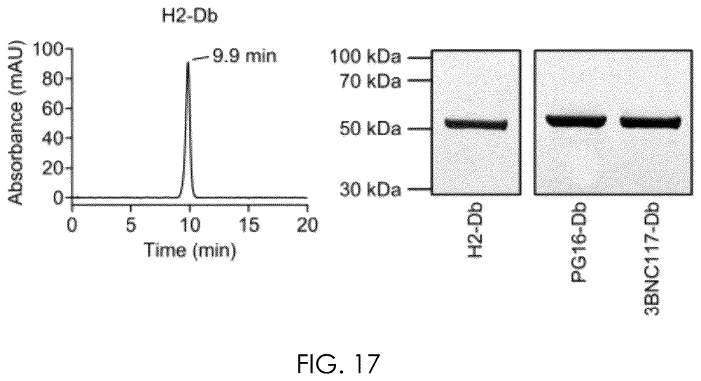

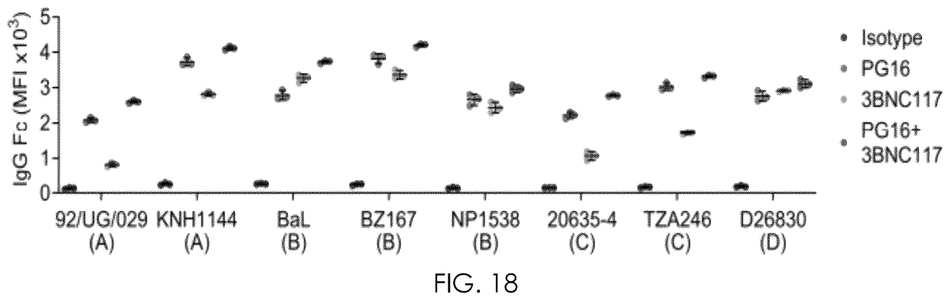

ScDbs induce robust NK cell activation. We incorporated sequences of antibodies against Env and CD16 into scDbs. ScDbs consist of a single polypeptide chain encoding the heavy- and light-chain variable regions of two antibodies separated by flexible glycine linkers, which assemble to form two distinct, functional, single-chain variable fragment (scFv) domains^{16,22} (FIG. 1A). Two HIV-1-specific scDbs were designed based on the broadly neutralizing antibodies (bNAbs) PG16 (ref. 23) and 3BNC117 (ref. 24), which are specific for the apex region and the CD4-binding site of Env, respectively (Table 1 and FIG. 17). Both bNAbs were reported to recognize diverse HIV-1 Env isolates and eliminate HIV-1-infected cells through ADCC25,26. PG16 and 3BNC117 were coupled with the CD16 antibody NM3E2 to generate PG16-Db and 3BNC117-Db (Table 1 and FIG. 17). We also generated an irrelevant isotype control scDb called H2-Db, which incorporates an antibody directed against a mutant p53 epitope presented on HLA-A*02:01 (ref. 22) combined with NM3E2. All three scDbs bound CD16 recombinant proteins (FIGS. 7A-7B) and cell-surface CD16 expressed on primary human NK cells (FIGS. 1B-1C and FIG. 7C-7D). H2-Db, PG16-Db and 3BNC117-Db did not bind to NK cells preincubated with a CD16-specific antibody or to B cells that lack CD16 expression (FIGS. 1B-1C and FIG. 7C-7D). In addition, PG16-Db and 3BNC117-Db bound Env presented on the surface of HEK293T cells transfected with env genes from a diverse set of HIV-1 isolates (FIG. 1D and FIG. 18), indicating that both HIV-1-specific scDbs retained the multiple HIV-1 subtype breadth of Env binding exhibited by their parental bNAbs. H2-Db did not bind transfected HEK293T cells. Thus, PG16-Db and 3BNC117-Db specifically bound HIV-1 Env and CD16 in the expected manner.

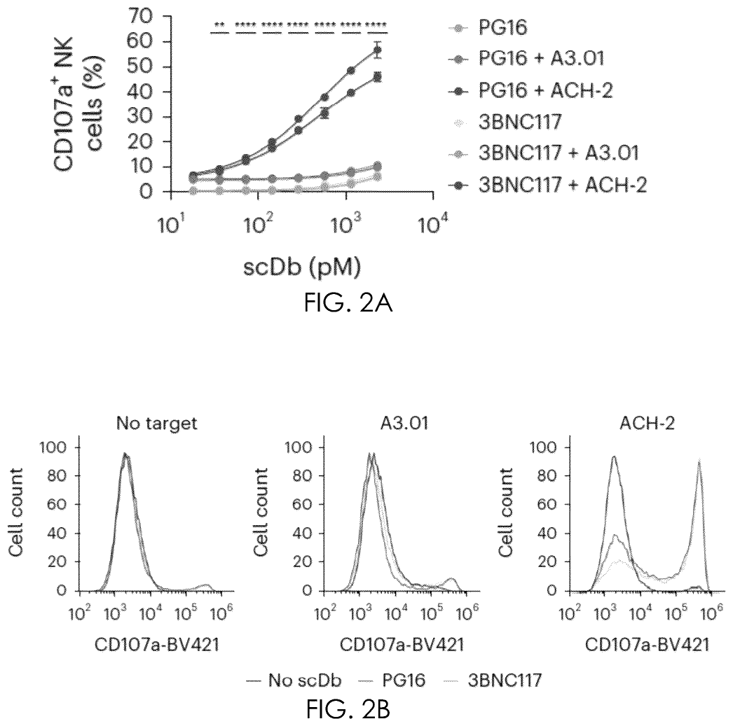

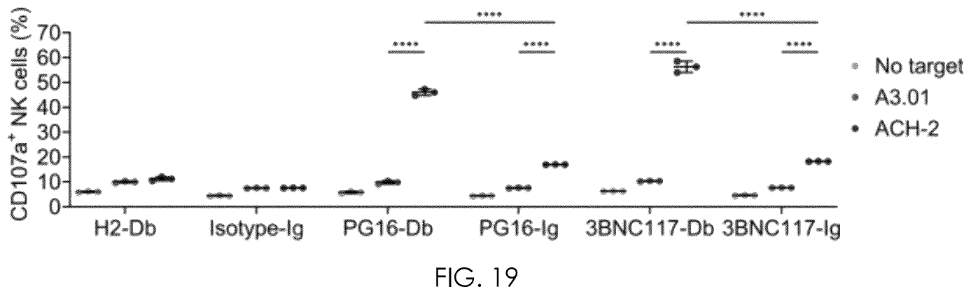

We next assessed the ability of the scDbs to induce specific activation of NK cells. NK cells from HIV-1-negative participants were cocultured for 6 h with tumor necrosis factor (TNF)-pretreated ACH-2 cells in the presence of PG16-Db or 3BNC117-Db. ACH-2 cells are transformed CD4+ T cells which carry an HIV-1 provirus that can be induced with TNF and have been used as a model of HIV-1 latency²⁷. Addition of PG16-Db or 3BNC117-Db resulted in dose-dependent increases in NK cell expression of CD107a, a marker of degranulation (FIG. 2A-2B). Neither PG16-Db nor 3BNC117-Db induced NK cell expression of CD107a in cocultures with the TNF-pretreated, uninfected parental T cell line A3.01 (FIG. 2A-2B), indicating improved cytolytic degranulation in response to the HIV-1-infected target cells. NK cell expression of CD107a was greatly enhanced by PG16-Db and 3BNC117-Db compared with equimolar concentrations of the respective parental IgG molecules, termed here PG16-Ig and 3BNC117-Ig, in NK cell cocultures with TNF-pretreated ACH-2 cells (FIG. 19). PG16-Db and 3BNC117-Db induced only minimal NK cell degranulation in the absence of target cells (FIG. 2A).

In addition, we assessed indicators of NK cell activation including cytolytic granule proteins (granzyme A, granzyme B, perforin and granulysin), a soluble TNF family death ligand (sFasL) and inflammatory cytokines (interferon (IFN)-γ, TNF) in the supernatants of the same cultures. At concentrations between 10 and 1,000 pM, PG16-Db and 3BNC117-Db elicited dose-dependent increases in supernatant concentrations of these effector molecules in the presence of HIV-1-infected (ACH-2), but not uninfected (A3.01), target cells (data not shown). Even at concentrations <300 pM, the scDbs induced higher release of all assayed effector molecules when cultured with ACH-2 cells than in the corresponding A3.01 cocultures (data not shown). Notably, PG16-Db and 3BNC117-Db did not increase the production of effector molecules in the absence of Env-expressing ACH-2 cells (data not shown). Together, these results indicated that PG16-Db and 3BNC117-Db potently induced polyfunctional NK cell activity in the presence of Env-expressing target cells.

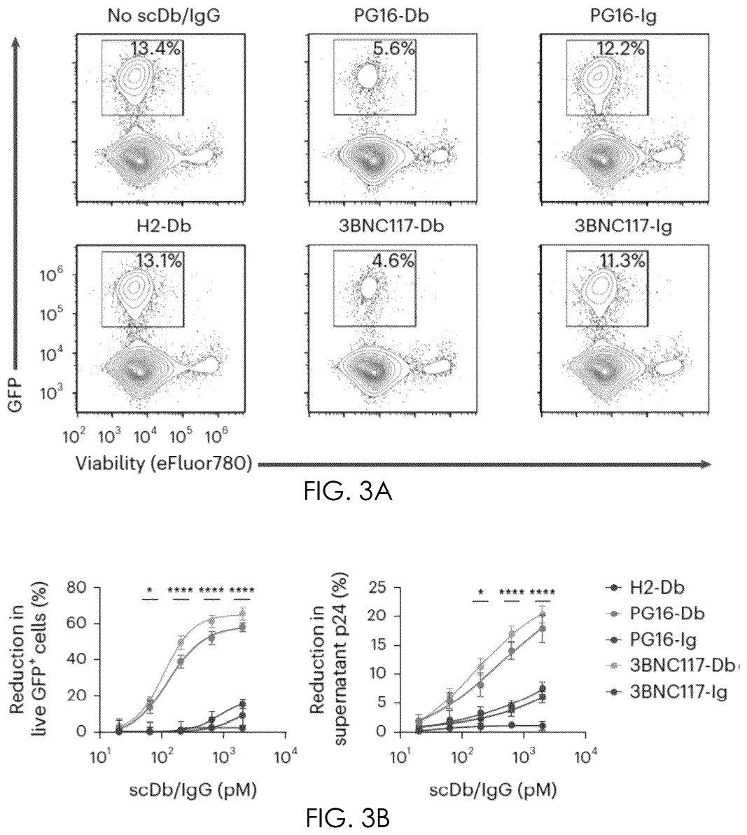

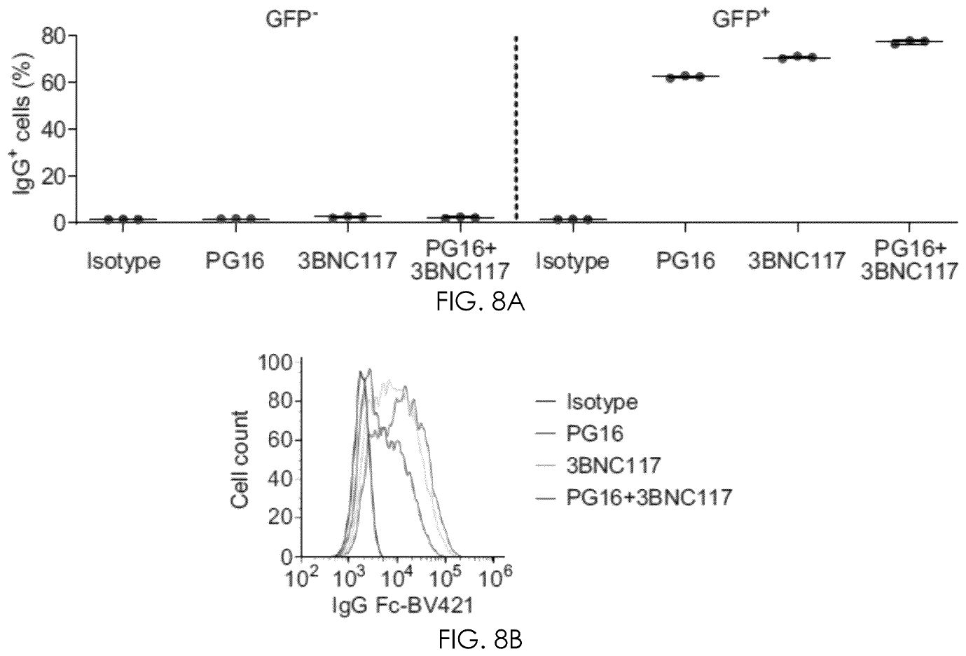

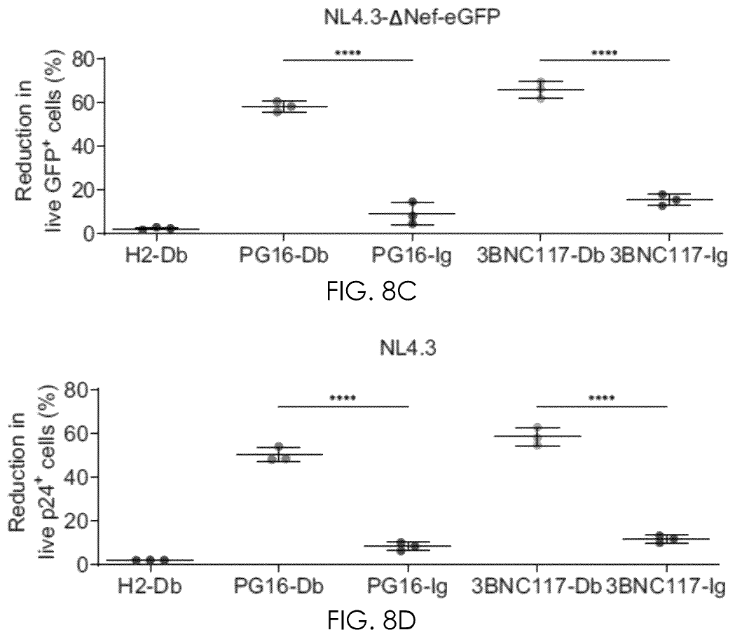

ScDbs induce NK cell-mediated elimination of HIV-1+ cells. To test the ability of the scDbs to promote killing of HIV-1-infected cells, CD4+ T cells from HIV-participants were activated with CD3+CD28 antibodies and infected with a replication-competent HIV-1 construct carrying enhanced green fluorescent protein (eGFP) in the open reading frame of the nef gene (NL4.3-ΔNef-eGFP). PG16-Ig and 3BNC117-Ig bound viable GFP+CD4+ T cells (FIG. 8A-8B), indicating that the infected CD4+ T cells expressed Env. Infected CD4+ T cells were then cocultured with autologous NK cells at an effector:target (E:T) ratio of 1:3 in the presence of the scDbs or their parental immunoglobulin G (IgG) molecules. After 18 h, we observed strong, dose-dependent decreases in viable GFP+CD4+ T cells with both PG16-Db and 3BNC117-Db compared with H2-Db or no scDb (FIGS. 3A-3B and FIG. 8C). At 2,000 pM, PG16-Db and 3BNC117-Db mediated 58% and 66% reductions in the number of viable GFP+ cells compared with no scDb treatment, respectively (FIG. 3B). Equimolar concentrations of PG16-Ig and 3BNC117-Ig resulted in 9% and 15% reductions in GFP+CD4+ T cells, respectively (FIG. 3B). There was no significant reduction in viable GFP+CD4+ T cells with H2-Db at any concentration tested (FIG. 3B). In addition, coculture of NK cells and autologous CD4+ T cells infected with a replication-competent HIV-1 construct that has a functional nef gene (NL4.3) resulted in 50% and 58% reductions in viable CD4+ T cells expressing HIV-1 p24 antigen with PG16-Db and 3BNC117-Db, respectively (FIG. 8D). At a concentration of 2,000 pM PG16-Db and 3BNC117-Db resulted in 18% and 20% decreases in p24 in the supernatant, respectively, compared with the no scDb control (FIG. 3B), consistent with the scDb-mediated elimination of HIV-1-infected cells. The reduction in supernatant p24 with H2-Db was negligible (1%), whereas those induced by PG16-Ig and 3BNC117-Ig were detectable (6% and 7%), but weaker than those induced by PG16-Db and 3BNC117-Db (FIG. 3B). The dose-response curves for viable GFP+CD4+ T cells and supernatant p24 indicated that PG16-Db and 3BNC117-Db were at least 1-2 log more potent than the corresponding IgG molecules in mediating the killing of HIV-1-infected CD4+ T cells (FIG. 3B), probably because the CD16-specific component of the scDbs had a much higher affinity for CD16 than the Fc domain of IgG molecules²⁸. These results showed that PG16-Db and 3BNC117-Db promoted efficient NK cell-mediated killing of HIV-1-infected CD4+ T cells and did so at much lower concentrations than the parental IgG molecules.

ScDbs induce ex vivo reduction of HIV-1 reservoirs. We next tested whether PG16-Db and 3BNC117-Db promoted the elimination of latently infected CD4+ T cells from people on long-term ART after latency reversal ex vivo. Peripheral blood mononuclear cells (PBMCs) were collected from 11 male participants (median age=56 years, range=34-69 years) with undetectable plasma HIV-1 RNA levels (<50 copies per ml) for >72 months (median time on ART=289 months, range=130-360 months; median time on suppressive ART=185 months, range=79-275 months; see Table 2 for ART regimens). Primary CD4+ T cells from two participants were stimulated with a panel of LRAs for 18 h, followed by coculture with autologous NK cells at an E:T ratio of 1:3 in the presence of PG16-Db, 3BNC117-Db or an equimolar combination of both for 18 h. CD4+ T cells were re-isolated and depleted of apoptotic cells by immunomagnetic selection of annexin-V+ cells to prevent measurement of HIV-1 DNA from cells killed during coculture. The frequency of intact provirus+ cells was measured by the intact proviral DNA assay (IPDA), a droplet digital (dd) PCR assay that quantifies proviruses that lack major defects, such as large internal deletions and APOBEC3−-mediated hypermutation. No significant reduction in intact provirus+ cells was observed in CD4+ T cells incubated with the no LRA control, the protein kinase C agonist bryostatin, the SMAC mimetic AZD5582 or the histone deacetylase (HDAC) inhibitors romidepsin and SAHA in the presence of PG16-Db, 3BNC117-Db or PG16-Db+3BNC117-Db compared with the H2-Db control (FIG. 4A). However, when CD4+ T cells were treated with agents that induce global T cell activation, such as CD3+CD28 antibodies or phorbol myristate acetate+ ionomycin (PMA+I), there were significant reductions of up to 56% and 67%, respectively, in the frequency of intact provirus+CD4+ T cells were attained with PG16-Db+3BNC117-Db relative to H2-Db (FIG. 4A).

To determine whether the reduction in intact provirus+CD4+ T cells was dependent on the degree of latency reversal, we performed paired measurements of cell-associated polyadenylated HIV-1 RNA (caRNA) in aliquots of LRA-treated CD4+ T cells before NK cell coculture. Of the six LRAs tested, only CD3+CD28 antibodies and PMA+I resulted in a significant increase in caRNA transcripts compared with the untreated control in both participants (FIG. 4B), consistent with prior reports²⁹. The HDAC inhibitor romidepsin elicited a significant increase in caRNA transcripts in one of two participants (designated as DEL-SPC-044), but the corresponding reductions in intact provirus+ cells relative to the H2-Db control (11% with PG16-Db, 17% with 3BNC117-Db and 16% with PG16-Db+3BNC117-Db) in this participant did not reach statistical significance (FIG. 4A). We found a significant correlation between caRNA induction and the reduction in intact provirus+ cells (FIG. 4C), suggesting that scDb-mediated elimination of intact provirus+ cells depended on efficient latency reversal.

Next, we tested whether PG16-Db and 3BNC117-Db mediated the elimination of HIV-1-infected cells under maximal latency reversal conditions ex vivo. We stimulated CD4+ T cells isolated from each of the 11 participants with PMA+I, cocultured them with autologous NK cells in the presence of PG16-Db, 3BNC117-Db or PG16-Db+3BNC117-Db for 18 h, re-isolated annexin-V-CD4+ T cells and used the IPDA to measure the frequency of intact provirus+ cells. The frequency of intact provirus+CD4+ T cells was significantly lower relative to the H2-Db control for five participants with PG16-Db, eight with 3BNC117-Db and nine with PG16-Db+3BNC117-Db (FIG. 5A). Staining of untreated PBMCs from eight participants indicated no significant correlation between the reduction in the frequency of intact provirus+CD4+ T cells and the expression of CD16, NKG2D, Siglec-7, CD57 or PD-1 (programmed cell death protein 1) on CD3-CD56+NK cells (FIG. 9), suggesting that the variability in response to scDb treatment did not correlate with NK cell phenotypic differences, at least as determined by these markers. Considering all participants, the PG16-Db, 3BNC117-Db and PG16-Db+3BNC117-Db resulted in 41%, 45% and 49% average reductions in the frequency of intact provirus+CD4+ T cells, respectively, when compared with H2-Db (FIG. 5A-5B).

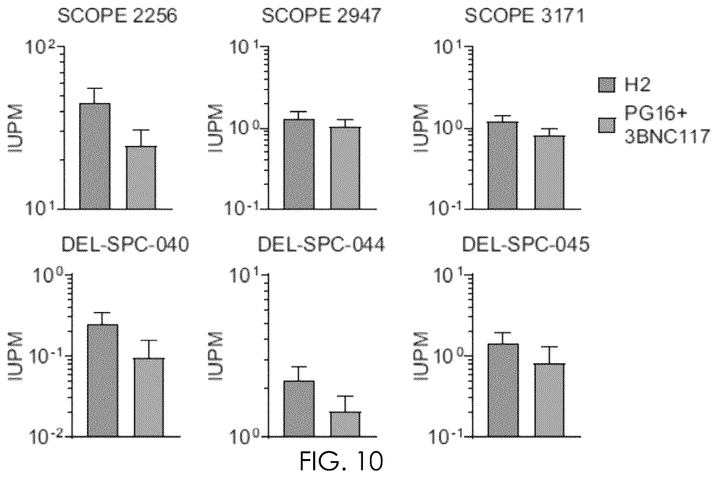

Only a subset of intact provirus+ cells can be induced to produce virus with a single or multiple rounds of stimulation³⁰. To better understand the effect of scDb treatment on the inducible population of reservoir cells, we plated the CD4+ T cells that survived coculture with autologous NK cells in the presence of PG16-Db and 3BNC117-Db at limiting dilution in quantitative viral outgrowth assays (QVOAs), which measure inducible, replication-competent proviruses. All six participants tested had reduced infectious units per million (IUPM) values when cocultures were treated with PG16-Db and 3BNC117-Db compared with H2-Db (FIG. 10). PG16-Db+3BNC117-Db resulted in a 40% reduction in IUPM on average compared with H2-Db (FIG. 5B).

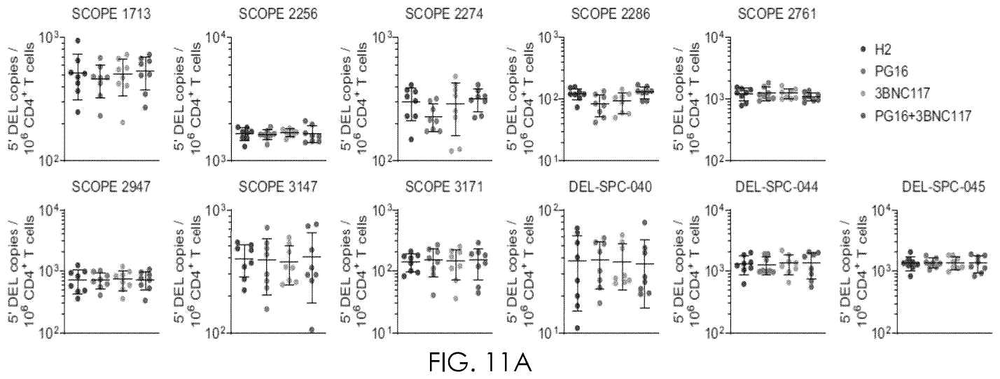

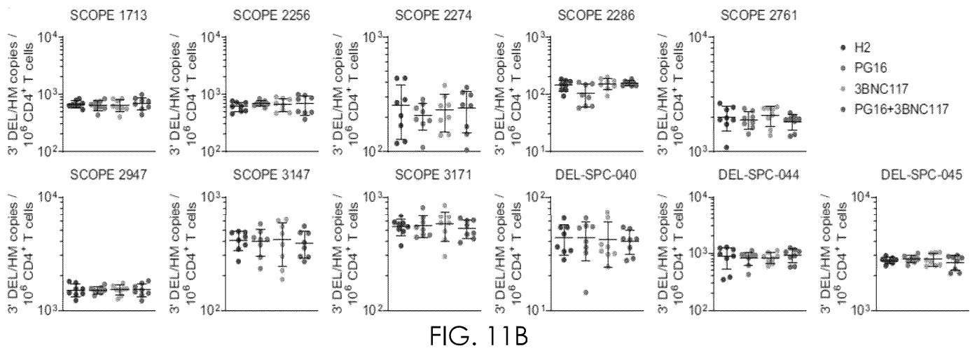

The IPDA measures intact HIV-1 proviruses but also proviruses with common defects, including large internal deletions and hypermutation, which make up the vast majority of all proviruses in people with HIV-1 (refs. 31,32). Most defective proviruses are defective in the tat gene, thereby preventing high levels of viral gene expression^{31,32}, and many have defects that affect the env gene, which prevent productive expression of Env protein and render the HIV-1-infected cells nondetectable by Env-specific antibodies. We observed no significant change in the frequency of CD4+ T cells with 5′- or 3′-defective proviruses in any of the 11 donors compared with H2-Db (FIG. 5B and FIG. 11A-11B), indicating that, in cultures in which intact provirus+CD4+ T cells were eliminated, HIV-1-infected cells with highly defective proviruses were not affected, providing a compelling internal control. Thus, the observed reduction in the frequency of intact provirus+CD4+ T cells was the result of scDb-mediated killing of HIV-1-infected cells that expressed high levels of Env. These results provide direct evidence that PG16-Db and 3BNC117-Db promote NK cell killing of latent reservoir cells from people on ART after ex vivo latency reversal.

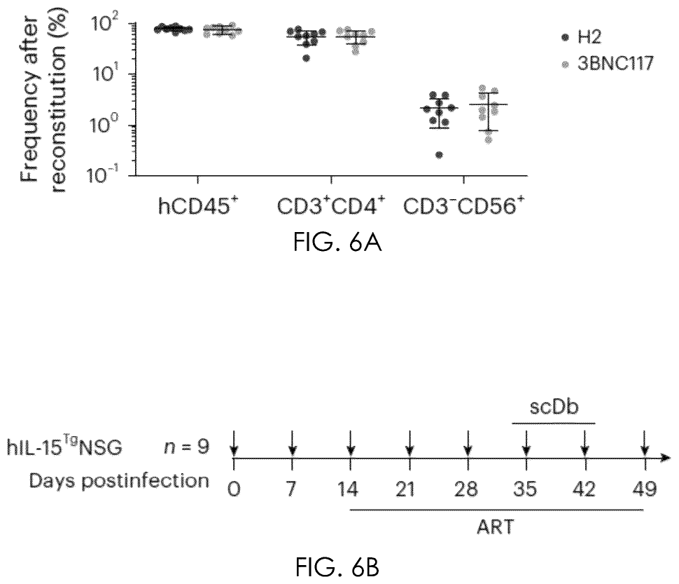

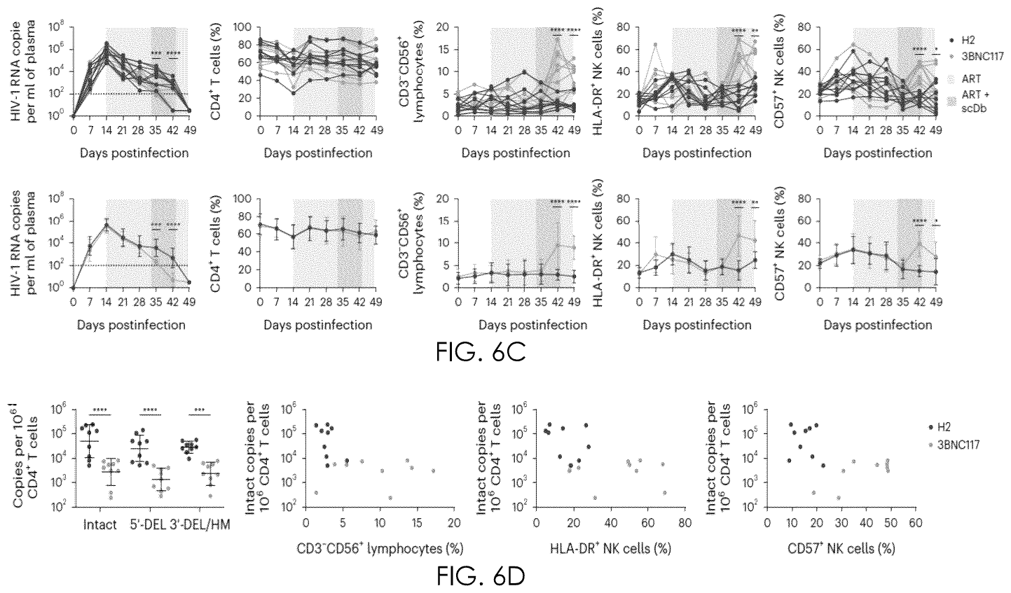

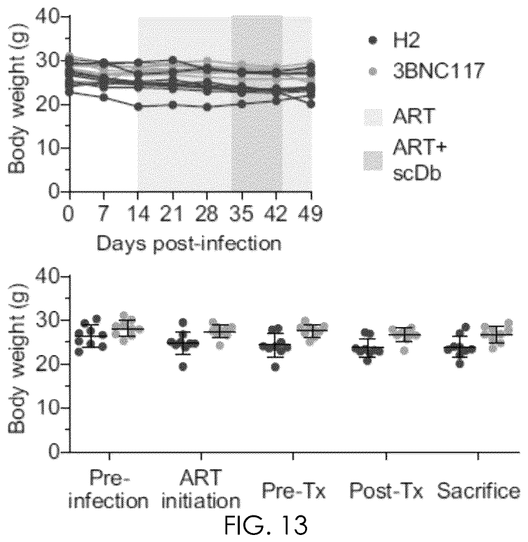

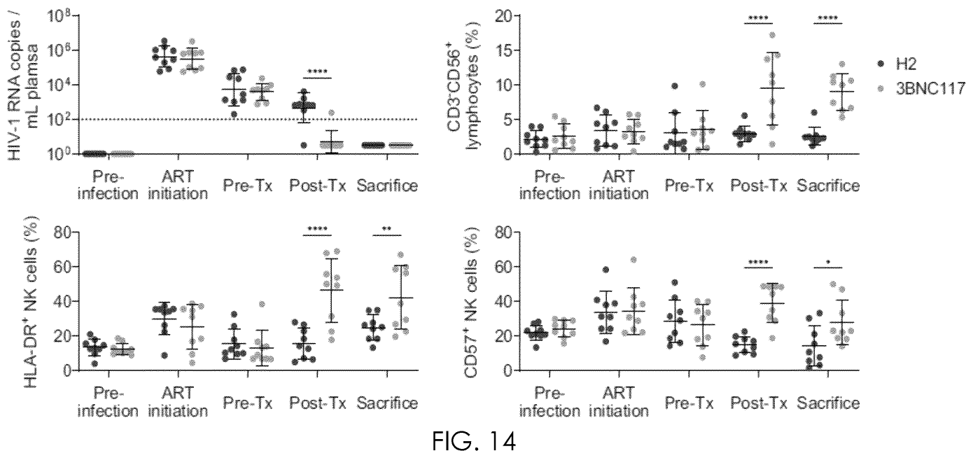

ScDb induces reduction of HIV-1+ cells in humanized mice. Next, we assessed the ability of one of the scDbs, 3BNC117-Db, to drive activation of NK cells and promote elimination of HIV-1-infected cells in humanized mice. Reconstitution of immunodeficient mice with human hematopoietic stem cells results in a reduced number of human NK cells with poor functional activity³³. However, transgenic expression of human interleukin-15 (hIL-15) in mice restores the development of human NK cells with functional cytolytic and tissue-homing properties^{34,35}. After reconstitution with hematopoietic stem cells, hIL-15 transgenic NOD.Cg-PrkdcscidIl2rgtm1Wjl mice (hereafter called hIL-15TgNSG) exhibited high frequencies of human CD4+ T cells and readily detectable human CD3-CD56+NK cells (FIG. 6A). The hIL-15TgNSG mice were infected with a transmitted/founder clone of HIV-1 (HIVSUMA), placed on ART to suppress viremia on day 14 post-infection, and administered 200 g of H2-Db (n=9) or 3BNC117-Db (n=9) through intraperitoneal injection daily for 10 d starting on day 33 post-infection (FIG. 6B), a protocol selected based on prior evaluation of the plasma pharmacokinetics of the scDbs after a single injection (FIG. 12). Mice were bled on days 0, 7, 14, 21, 28, 35, 42 and 49 post-infection to collect plasma and PBMCs, and were euthanized on day 49 post-infection for tissue collection. We did not observe evidence of toxicity or weight loss after scDb administration (FIG. 13), suggesting that H2-Db and 3BNC117-Db were tolerated at the administered dose. Plasma viral load had reached an average of 3.67×10⁵ HIV-1 RNA copies per ml at the time of ART initiation (day 14 post-infection) and decayed to an average of 4.48×10³ HIV-1 RNA copies per ml by day 28 post-infection (FIG. 6C and FIG. 14). As a result of the cytopathic effects of the virus during acute HIV-1 infection, the frequency of CD4+ T cells declined by 13% during the first 14 d post-infection (FIG. 6C). ART initiation resulted in recovery and stabilization of CD4+ T cell frequencies (FIG. 6C). No differences in plasma viral load or frequency of CD4+ T cells were detected between H2-Db- and 3BNC117-Db-treated mice at any timepoint before scDb administration. Treatment with 3BNC117-Db resulted in a faster decline of plasma HIV-1 RNA apparent at days 35 and 42 post-infection compared with H2-Db (FIG. 6C). By day 42, eight of nine mice that received 3BNC117-Db had attained suppression of plasma HIV-1 RNA to below the limit of detection compared with one of nine mice that received H2-Db (FIG. 6C and FIG. 14).

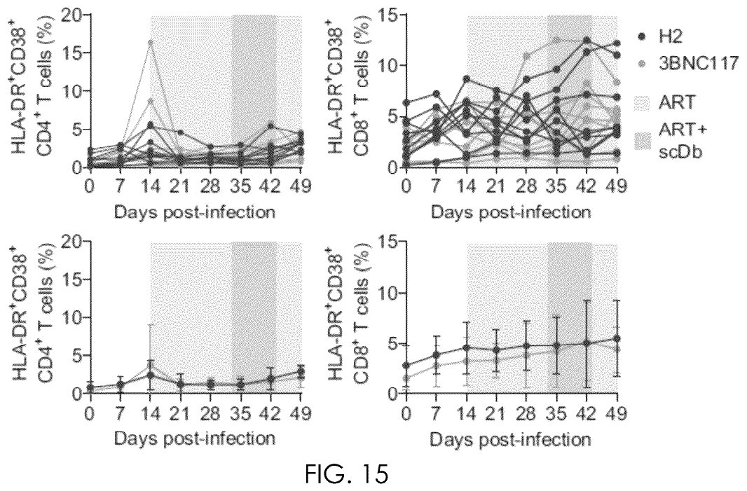

We then examined the effect of 3BNC117-Db on peripheral blood NK cells by quantifying the expansion of total CD3-CD56+NK cells within the CD45+ lymphocytes and the expression of human leukocyte antigen (HLA)-DR, an activation marker, and CD57, an NK cell maturation marker associated with superior NK cell cytolytic and cytokine-secreting functions³⁶. The frequencies of HLA-DR+NK cells and CD57+NK cells increased over the first 14 d post-infection (FIG. 6C and FIG. 14), probably corresponding to the emergence of NK cell responses against HIV-1 during acute infection. The increase in HLA-DR+NK cells and CD57+NK cells was transient, and their frequencies declined to near baseline levels after ART initiation (FIG. 6C and FIG. 14). After scDb administration, the frequency of peripheral blood CD3-CD56+NK cells was on average 3.2-fold higher in mice that received 3BNC117-Db compared with mice that received H2-Db at day 42 post-infection (FIG. 6C and FIG. 14), whereas the frequencies of HLA-DR+NK cells and CD57+NK cells increased 3.0-fold and 2.6-fold on average, respectively, in mice that received 3BNC117-Db compared with mice that received H2-Db at day 42 post-infection (FIG. 6C). The increased frequencies of total CD3-CD56+NK cells, HLA-DR+NK cells and CD57+NK cells in the 3BNC117-Db-treated mice waned slightly after cessation of scDb administration at day 43 post-infection, but remained significantly elevated compared with H2-Db-treated mice at day 49 (FIG. 6C and FIG. 14). We found no significant differences in the frequencies of CD4+ or CD8+ T cells that expressed the activation markers HLA-DR or CD38 during or after scDb administration in 3BNC117-Db- and H2-Db-treated mice (FIG. 15), indicating that 3BNC117-Db did not result in global T cell activation.

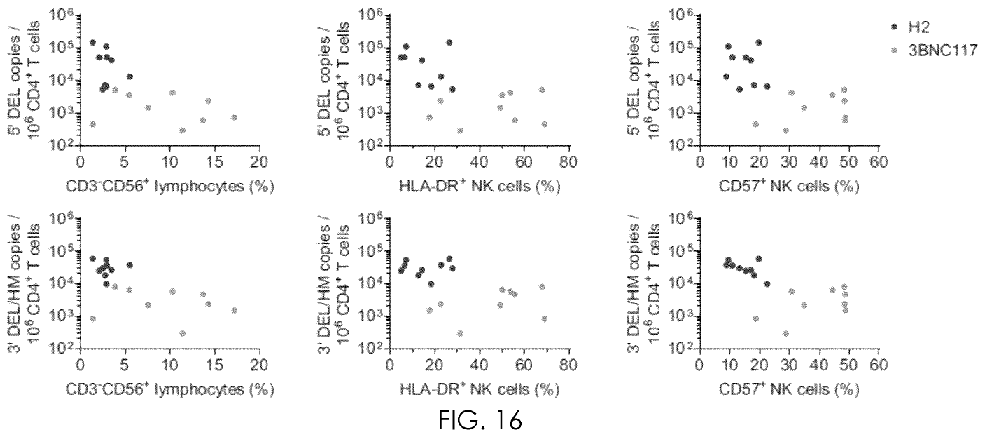

Last, IPDA quantification of HIV-1 reservoir size on DNA extracted from splenocytes at day 49 post-infection, when all mice had reached undetectable plasma HIV-1 RNA levels, showed that 3BNC117-Db-treated mice had a 25-fold average reduction in the frequency of intact provirus+CD4+ T cells, a 23-fold average reduction in the frequency of CD4+ T cells harboring proviruses with 5′-deletion and a 9-fold average reduction in CD4+ T cells harboring proviruses with 3′-deletion or hypermutation compared with H2-Db-treated mice (FIG. 6D). Mice treated with 3BNC117-Db also exhibited increased frequencies of CD3-CD56+NK cells, and higher fractions of HLA-DR+NK cells and CD57+NK cells posttreatment (day 42) relative to mice treated with H2-Db (FIG. 6D and FIG. 16). Thus, 3BNC117-Db promoted enhanced NK cell-mediated elimination of infected CD4+ T cells, which resulted in a smaller reservoir size at the time of complete ART-mediated viral suppression.

### Discussion

In the present study, we developed two bispecific antibodies that target Env and CD16 to enhance HIV-1-specific NK cell activity. PG16-Db and 3BNC117-Db promoted highly potent and specific NK cell activation and NK cell-mediated killing of HIV-1-infected cells. These bispecific antibodies mediated the elimination of up to 72% of intact provirus+CD4+ T cells from people on suppressive ART ex vivo, thus representing promising new therapeutics for HIV-1 reservoir reduction strategies. In addition, our evaluation of 3BNC117-Db in HIV-1-infected humanized mice suggested that bispecific antibodies may also have potential application for use in limiting reservoir establishment during acute HIV-1 infection or reducing reservoir size during chronic HIV-1 infection.

By using a high-affinity CD16 scFv, we developed scDbs that elicit stronger NK cell activation and killing of infected cells compared with parental IgG bNAbs. PG16-Db and 3BNC117-Db elicited HIV-1-specific NK cell activity and elimination of HIV-1-infected CD4+ T cells in vitro, at levels comparable to those of previously reported Env-CD16-bispecific antibodies^{37,38}. However, unlike previously reported Env-CD16-bispecific antibodies, the scDbs described in the present study were not dependent on prestimulation of the NK cell effectors with IL-2 or IL-15. In addition, scDb binding to CD16 alone did not result in notable NK cell activation or cytolysis of uninfected cells, an important consideration for in vivo applications. ScDb-induced activation of other CD16-expressing immune effectors such as neutrophils, macrophages and 76 T cells may occur, representing a possible safety concern in vivo, but also potentially allowing for further enhancement of infected CD4+ T cell clearance.

Ongoing viral replication reduces the fraction of cytotoxic CD56dimCD16+ NK cells and promotes the emergence of dysfunctional NK cell responses^{39,40}. However, ART can result in a partial or complete restoration of the CD56dimCD16+NK cell subset and NK cell activity^{39,41,42}. We did not find a relationship between scDb-enhanced NK cell cytotoxicity ex vivo and NK cell phenotype in the ART-treated people studied here.

The present study and others²⁹ have shown that single LRA treatments cause only small increases in HIV-1 caRNA transcripts. Consequently, none of the individual LRAs tested was probably able to induce high levels of Env expression in most intact provirus+ cells, thereby limiting elimination of those cells when paired with autologous NK cell coculture. Env surface density is a determinant of infected cell elimination for other Env-specific immunotherapeutic approaches, including bNAb-based CAR T cells⁴³. To achieve robust elimination of reservoir cells in vivo with Env-directed therapeutics, single or combinations of LRAs with greater potency will be needed.

The efficient elimination of intact provirus+ cells across multiple participants suggested that PMA+I treatment resulted in sufficient Env expression for scDb-mediated elimination. We did not observe a reduction in cells that harbored proviruses with deletions in env or extensive hypermutation in any participant, because such cells are not able to express Env. Cells that harbored proviruses with 5′-deletions also escaped elimination. Many of these proviruses have defects that affect the major splice donor site (MSD)31,32. As the mRNA encoding Env is generated by a splicing reaction involving the MSD site, MSD defects markedly reduce Env expression44 and may prevent targeting by scDbs.

By combining PG16-Db and 3BNC117-Db, which target nonoverlapping Env epitopes, we observed a slightly greater average elimination of reservoir cells than either single scDb mediated at the same concentration. This may reflect either the improved breadth of viral variant recognition attainable with multiple bNAbs or synergy due to simultaneous binding to multiple epitopes on Env. To achieve more potent and universally effective elimination of reservoir cells, future efforts should explore combinations of targeting modalities. The scDbs described in the present study may complement CD8+ T cell-based antibodies¹⁴⁻¹⁶ by engaging multiple cytolytic immune cell types. In addition, bispecific antibodies that target HIV-1 peptide-major histocompatibility complexes¹⁶ may synergize with Env-targeted approaches by enabling the elimination of reservoir cells with suboptimal Env expression.

A limitation of our humanized mouse model is that the reduction in infected CD4+ T cells probably reflects the elimination of productively infected CD4+ T cells, which comprise most of the infected cell population during the initial stages of HIV-1 infection rather than the elimination of latently infected CD4+ T cells. Future nonhuman primate studies, in which a combination treatment of scDbs and LRAs is administered in the context of long-term viral suppression, will be necessary to directly evaluate scDb-mediated clearance of latent reservoir cells. Despite this caveat, these findings suggest that scDbs may have potential clinical applications during acute HIV-1 infection at the time of ART initiation. NK cell functional activity during acute HIV-1 infection correlates with viral control⁴⁵⁻⁴⁶ and less severe disease progression47 during chronic infection. High frequencies of CD57+NK cells during acute HIV-1 infection have been associated with lower viral load after 3 months on ART and faster time to viral suppression⁴⁸. In addition, the CD57+NK cell subset exhibits highly potent ADCC and cytokine-producing activity in people with HIV-1 (ref. 36). Thus, the potential for 3BNC117-Db to promote elimination of reservoir cells during early HIV-1 infection warrants further exploration.

The short half-life of the scDbs in humanized mice probably resulted from the absence of Fc domains which, through interaction with the neonatal Fc receptor, extended the half-life of traditional monoclonal IgG-based therapeutics. The fast decay rate of scDbs in plasma may be a challenge for clinical application, because daily injections or continuous infusion will probably be necessary to maintain therapeutic concentrations. ScDbs may benefit from advances in protein engineering aimed at extending the half-life of small recombinant antibody molecules in vivo^{49,50}. In addition, as the scDbs are engineered constructs with features that do not resemble naturally occurring antibodies, the emergence of scDb-specific antibody responses that could affect clinical treatment efficacy may occur. Although we did not find evidence of induction of global immune activation, a detailed profiling of cytokine responses induced by administration of scDbs in vivo will be necessary to alleviate safety concerns over the potential for cytokine-related adverse reactions.

Taken together, PG16-Db and 3BNC117-Db deserve additional evaluation as therapeutic interventions for HIV-1 reservoir reduction. We expect future studies to focus on combining these scDbs with other treatments to further improve on attainable clearance of the HIV-1 reservoir cells.

TABLE 1
Sequences of the V_{H} and V_{L} regions for the relevant scDbs.

| scFv | Antigen | VH | VL |
|---|---|---|---|
| H2 | Human p53^{R175h} | EVQLVESGGGLVQPGGSLRL | DIQMTQSPSSLSASVGDRVTI |
| | pHLA-A2 | SCAASGFNVYASGMHWVRAQ | TCRASQDVNTAVAWYQQKPGK |
| | | APGKGLEWVAKIYPDSDYTY | APKLLIYSAYFLYSGVPSRFS |
| | | YADSVKGRFTISADTSKNTA | GSRSGTDFTLTISSLQPEDFA |
| | | YLQMNSLRAEDTAVYYCSRD | TYYCQQYSRYSPVTFGQGTKV |
| | | SSFYYVYAMDYWGQGTLVTV | EIK (SEQ ID NO: 26) |
| | | SS (SEQ ID NO: 25) | |
| PG16 | HIV-1 Env V1/V2 | QEQLVESGGGVVQPGGSLRL | QSALTQPASVSGSPGQTITIS |
| | | SCLASGFTFHKYGMHWVRQA | CNGTSSDVGGFDSVSWYQQSP |
| | | PGKGLEWVALISDDGMRKYH | GKAPKVMVFDVSHRPSGISNR |
| | | SDSMWGRVTISRDNSKNTLY | FSGSKSGNTASLTISGLHIED |
| | | LQFSSLKVEDTAMFFCAREA | EGDYFCSSLTDRSHRIFGGGT |
| | | GGPIWHDDVKYYDENDGYYN | KVTVL (SEQ ID NO: 5) |
| | | YHYMDVWGKGTTVTVSS | |
| | | (SEQ ID NO: 1) | |
| 3BNC117 | HIV-1 Env | QVQLLQSGAAVTKPGASVRV | DIQMTQSPSSLSASVGDTVTI |
| | CD4bs | SCEASGYNIRDYFIHWWRQA | TCQANGYLNWYQQRRGKAPKL |
| | | PGQGLQWVGWINPKTGQPNN | LIYDGSKLERGVPSRFSGRRW |
| | | PRQFQGRVSLTRHASWDFDT | GQEYNLTINNLQPEDIATYFC |
| | | FSFYMDLKALRSDDTAVYFC | QVYEFVVPGTRLDLK |
| | | ARQRSDYWDFDVWGSGTQVT | (SEQ ID NO: 13) |
| | | VSS (SEQ ID NO: 9) | |
| NM3E2 | Human CD16 | EVQLVESGGGVVRPGGSLRL | SSELTQDPAVSVALGQTVRIT |
| | | SCAASGFTFDDYGMSWVRQA | CQGDSLRSYYASWYQQKPGQA |
| | | PGKGLEWVSGINWNGGSTGY | PVLVIYGKNNRPSGIPDRFSG |
| | | ADSVKGRFTISRDNAKNSLY | SSSGNTASLTITGAQAEDEAD |
| | | LQMNSLRAEDTAVYYCARGR | YYCNSRDSSGNHVVFGGGTKL |
| | | SLLFDYWGQGTLVTVSR | TVG (SEQ ID NO: 21) |
| | | (SEQ ID NO: 17) | |

Underlined: Complementarity Determining Regions (CDRs)
PG16 VH CDRs 1-3: SGFTFHKYGMH (SEQ ID NO: 2); ISDDGMRKYHSDSMW (SEQ ID NO: 3); EAGGPIWHDDVKYYDFNDGYYNYHYMDV (SEQ ID NO: 4)
PG16 VL CDRs 1-3: NGTSSDVGGFDSVS (SEQ ID NO: 6); DVSHRPSG (SEQ ID NO: 7); SSLTDRSHRI (SEQ ID NO: 8)
3BNC117 VH CDRs 1-3: DYFIH (SEQ ID NO: 10); WINPKTGQPNNPRQFQG (SEQ ID NO: 11); QRSDYWDFDV (SEQ ID NO: 12)
3BNC117 VH CDRs 1-3: QANGYLN (SEQ ID NO: 14); DGSKLER (SEQ ID NO: 15); QVYEF (SEQ ID NO: 16)
NME3E2 VH CDRs 1-3: DYGMS (SEQ ID NO: 18); GINWNGGSTGYADSVKG (SEQ ID NO: 19); GRSLLFDY (SEQ ID NO: 20)
NME3E2 VL CDRs 1-3: QGDSLRSYYAS (SEQ ID NO: 22); GKNNRPS (SEQ ID NO: 23); NSRDSSGNHVV (SEQ ID NO: 24)

TABLE 2
Characteristics of study participants.

| | | | | Time | | | Time on |
|---|---|---|---|---|---|---|---|
| | | | | since | | Time on | suppressive |
| Participant | | | | diagnosis | | ART | ART |
| ID | Age | Sex | Race | (months) | ART regimen | (months) | (months) |
| SCOPE | 37 | M | White | 130 | FTC/TAF/BIC | 130 | 124 |
| 1713 | | | | | | | |
| SCOPE | 66 | M | White | 437 | FTC/TAF/RPV/TCV | 347 | 248 |
| 2256 | | | | | | | |
| SCOPE | 60 | M | White | 235 | FTC/TAF/NVP | 222 | 220 |
| 2274 | | | | | | | |
| SCOPE | 56 | M | White | 233 | FTC/TAF/RPV | 233 | 229 |
| 2286 | | | | | | | |
| SCOPE | 49 | M | Hispanic | 289 | FTC/TAF/BIC | 289 | 275 |
| 2761 | | | | | | | |
| SCOPE | 56 | M | Hispanic | 368 | 3TC/ABC/TDF/NVP | 344 | 79 |
| 2947 | | | | | | | |
| SCOPE | 65 | M | Hispanic | 342 | FTC/TAF/BIC | 320 | 172 |
| 3147 | | | | | | | |
| SCOPE | 69 | M | Mixed | 329 | FTC/TAF/RGV/DRV/RTV | 326 | 193 |
| 3171 | | | | | | | |
| DEL-SPC- | 34 | M | Mixed | 153 | 3TC/ABC/TCV | 146 | 135 |
| 040 | | | | | | | |
| DEL-SPC- | 55 | M | AA | 238 | FTC/TAF/BIC | 216 | 176 |
| 044 | | | | | | | |
| DEL-SPC- | 68 | M | White | 384 | FTC/TDF/ATV/RTV | 360 | 86 |
| 045 | | | | | | | |

Abacavir, ABC; African American, AA; antiretroviral therapy, ART; atazanavir, ATV; bictegravir, BIC; darunavir, DRV; dolutegravir, TCV; emtricitabine, FTC; lamivudine, 3TC; nevirapine, NVP; raltegravir, RGV; rilpivirine, RPV; ritonavir, RTV; tenofovir alafenamide, TAF; tenofovir disoproxil fumarate, TDF.

### REFERENCES

1. Perelson, A. S. et al. Decay characteristics of HIV-1-infected compartments during combination therapy. Nature 387, 188-191 (1997).
2. Vittinghoff, E. et al. Combination antiretroviral therapy and recent declines in AIDS incidence and mortality. J. Infect. Dis. 179, 717-720 (1999).
3. Chun, T. W. et al. In vivo fate of HIV-1-infected T cells: quantitative analysis of the transition to stable latency. Nat. Med. 1, 1284-1290 (1995).
4. Chun, T.-W. et al. Quantification of latent tissue reservoirs and total body viral load in HIV-1 infection. Nature 387, 183-188 (1997).
5. Sengupta, S. & Siliciano, R. F. Targeting the latent reservoir for HIV-1. Immunity 48, 872-895 (2018).
6. Board, N. L., Moskovljevic, M., Wu, F., Siliciano, R. F. & Siliciano, J. D. Engaging innate immunity in HIV-1 cure strategies. Nat. Rev. Immunol. 22, 499-512 (2022).
7. Archin, N. M. et al. Administration of vorinostat disrupts HIV-1 latency in patients on antiretroviral therapy. Nature 487, 482-485 (2012).
8. Rasmussen, T. A. et al. Panobinostat, a histone deacetylase inhibitor, for latent-virus reactivation in HIV-infected patients on suppressive antiretroviral therapy: a phase 1/2, single group, clinical trial. Lancet HIV 1, e13-e21 (2014).
9. Søgaard, O. S. et al. The depsipeptide romidepsin reverses HIV-1 latency in vivo. PLoS Pathog. 11, 1-22 (2015).
10. Day, C. L. et al. PD-1 expression on HIV-specific T cells is associated with T-cell exhaustion and disease progression. Nature 443, 350-354 (2006).
11. Chew, G. M. et al. TIGIT marks exhausted T cells, correlates with disease progression, and serves as a target for immune restoration in HIV and SIV infection. PLoS Pathog. 12, e1005349 (2016).
12. Deng, K. et al. Broad CTL response is required to clear latent HIV-1 due to dominance of escape mutations. Nature 517, 381-385 (2015).
13. Labrijn, A. F., Janmaat, M. L., Reichert, J. M. & Parren, P. W. H. I. Bispecific antibodies: a mechanistic review of the pipeline. Nat. Rev. Drug Discov. 18, 585-608 (2019).
14. Sung, J. A. M. et al. Dual-affinity re-targeting proteins direct T cell-mediated cytolysis of latently HIV-infected cells. J. Clin. Invest. 125, 4077-4090 (2015).
15. Pegu, A. et al. Activation and lysis of human CD4 cells latently infected with HIV-1. Nat. Commun. 6, 1-9 (2015).
16. Sengupta, S. et al. TCR-mimic bispecific antibodies to target the HIV-1 reservoir. Proc. Natl Acad. Sci. USA 119, e2123406119 (2022).
17. Jones, R. B. et al. Histone deacetylase inhibitors impair the elimination of HIV-infected cells by cytotoxic T-lymphocytes. PLoS Pathog. 10, e1004287 (2014).
18. Walker-Sperling, V. E., Pohlmeyer, C. W., Tarwater, P. M. & Blankson, J. N. The effect of latency reversal agents on primary CD8+ T cells: implications for shock and kill strategies for human immunodeficiency virus eradication. eBioMedicine 8, 217-229 (2016).
19. Kwaa, A. K. et al. The effect of Ingenol-B on the suppressive capacity of elite suppressor HIV-specific CD8+ T cells. PLoS ONE 12, e0174516 (2017).
20. Nagorsen, D., Kufer, P., Baeuerle, P. A. & Bargou, R. Blinatumomab: a historical perspective. Pharmacol. Ther. 136, 334-342 (2012).
21. Hosseini, I. et al. Mitigating the risk of cytokine release syndrome in a Phase I trial of CD20/CD3 bispecific antibody mosunetuzumab in NHL: impact of translational system modeling. npj Syst. Biol. Appl. 6, 28 (2020).
22. Hsiue, E. H.-C. et al. Targeting a neoantigen derived from a common TP53 mutation. Science 371, eabc8697 (2021).
23. Walker, L. M. et al. Broad and potent neutralizing antibodies from an African donor reveal a new HIV-1 vaccine target. Science 326, 285-289 (2009).
24. Scheid, J. F. et al. Sequence and structural convergence of broad and potent HIV antibodies that mimic CD4 binding. Science 333, 1633-1637 (2011).
25. Bruel, T. et al. Elimination of HIV-1-infected cells by broadly neutralizing antibodies. Nat. Commun. 7, 10844 (2016).
26. Ren, Y. et al. Relationships between neutralization, binding, and ADCC of broadly neutralizing antibodies against reservoir HIV. J. Virol. 95, e01808-e01820 (2020).
27. Folks, T. M. et al. Tumor necrosis factor alpha induces expression of human immunodeficiency virus in a chronically infected T-cell clone. Proc. Natl Acad. Sci. USA 86, 2365-2368 (1989).
28. de Taeye, S. W. et al. FcγR binding and ADCC activity of human IgG allotypes. Front. Immunol. 11, 740 (2020).
29. Bullen, C. K., Laird, G. M., Durand, C. M., Siliciano, J. D. & Siliciano, R. F. New ex vivo approaches distinguish effective and ineffective single agents for reversing HIV-1 latency in vivo. Nat. Med. 20, 425-429 (2014).
30. Kwon, K. J. et al. Different human resting memory CD4+ T cell subsets show similar low inducibility of latent HIV-1 proviruses. Sci. Transl. Med. 12, eaax6795 (2020).
31. Ho, Y.-C. et al. Replication-competent noninduced proviruses in the latent reservoir increase barrier to HIV-1 cure. Cell 155, 540-551 (2013).
32. Bruner, K. M. et al. Defective proviruses rapidly accumulate during acute HIV-1 infection. Nat. Med. 22, 1043-1049 (2016).
33. André, M. C. et al. Long-term human CD34+ stem cell-engrafted nonobese diabetic/SCID/IL-2R gamma(null) mice show impaired CD8+ T cell maintenance and a functional arrest of immature NK cells. J. Immunol. 185, 2710-2720 (2010).
34. Herndler-Brandstetter, D. et al. Humanized mouse model supports development, function, and tissue residency of human natural killer cells. Proc. Natl Acad. Sci. USA 114, E9626-E9634 (2017).
35. Sungur, C. M. et al. Human NK cells confer protection against HIV-1 infection in humanized mice. J. Clin. Invest. 132, e162694 (2022).
36. Tomescu, C. et al. Identification of the predominant human NK cell effector subset mediating ADCC against HIV-infected targets coated with BNAbs or plasma from PLWH. Eur. J. Immunol. 51, 2051-2061 (2021).
37. Pollara, J. et al. Redirection of cord blood T cells and natural killer cells for elimination of autologous HIV-1-infected target cells using bispecific DART® molecules. Front. Immunol. 11, 1-17 (2020).
38. Ramadoss, N. S. et al. Enhancing natural killer cell function with gp41-targeting bispecific antibodies to combat HIV infection. AIDS 34, 1313-1323 (2020).
39. Mavilio, D. et al. Natural killer cells in HIV-1 infection: dichotomous effects of viremia on inhibitory and activating receptors and their functional correlates. Proc. Natl Acad. Sci. USA 100, 15011-15016 (2003).
40. Mavilio, D. et al. Characterization of CD56−/CD16+ natural killer (NK) cells: a highly dysfunctional NK subset expanded in HIV-infected viremic individuals. Proc. Natl Acad. Sci. USA 102, 2886-2891 (2005).
41. Barker, E., Martinson, J., Brooks, C., Landay, A. & Deeks, S. Dysfunctional natural killer cells, in vivo, are governed by HIV viremia regardless of whether the infected individual is on antiretroviral therapy. AIDS 21, 2363-2365 (2007).
42. Luo, Z. et al. Increased natural killer cell activation in HIV-infected immunologic non-responders correlates with CD4+ T cell recovery after antiretroviral therapy and viral suppression. PLoS ONE 12, e0167640 (2017).
43. Herzig, E. et al. Attacking latent HIV with convertibleCAR-T cells, a highly adaptable killing platform. Cell 179, 880-894.e10 (2019).
44. White, J. A. et al. Clonally expanded HIV-1 proviruses with 5′-leader defects can give rise to nonsuppressible residual viremia. J. Clin. Invest. 133, e165245 (2023).
45. Chen, X. et al. The early antibody-dependent cell-mediated cytotoxicity response is associated with lower viral set point in individuals with primary HIV infection. Front. Immunol. 9, 2322 (2018).
46. Kazer, S. W. et al. Integrated single-cell analysis of multicellular immune dynamics during hyperacute HIV-1 infection. Nat. Med. 26, 511-518 (2020).
47. Jiao, Y. et al. Short Communication: Longitudinal changes in peripheral blood NK cells during the first year of HIV-1 infection in CD4 low and CD4 high patient groups. AIDS Res. Hum. Retroviruses 31, 229-236 (2015).
48. Gondois-Rey, F. et al. A mature NK profile at the time of HIV primary infection is associated with an early response to cART. Front. Immunol. 8, 54 (2017).
49. Stork, R., Campigna, E., Robert, B., Müller, D. & Kontermann, R. E. Biodistribution of a bispecific single-chain diabody and its half-life extended derivatives. J. Biol. Chem. 284, 25612-25619 (2009).
50. Unverdorben, F., Farber-Schwarz, A., Richter, F., Hutt, M. & Kontermann, R. E. Half-life extension of a single-chain diabody by fusion to domain B of staphylococcal protein A. Protein Eng. Des. Sel. 25, 81-88 (2012).
51. Bruner, K. M. et al. A quantitative approach for measuring the reservoir of latent HIV-1 proviruses. Nature 566, 120-125 (2019).
52. Simonetti, F. R. et al. Intact proviral DNA assay analysis of large cohorts of people with HIV provides a benchmark for the frequency and composition of persistent proviral DNA. Proc. Natl Acad. Sci. USA 117, 18692-18700 (2020).
53. Laird, G. M. et al. Rapid quantification of the latent reservoir for HIV-1 using a viral outgrowth assay. PLoS Pathog. 9, e1003398 (2013).
54. Hong, F. et al. Novel assays for measurement of total cell-associated HIV-1 DNA and RNA. J. Clin. Microbiol. 54, 902-911 (2016).
55. Wiegand, A. et al. Single-cell analysis of HIV-1 transcriptional activity reveals expression of proviruses in expanded clones during ART. Proc. Natl Acad. Sci. USA 114, E3659-E3668 (2017).
56. Yuan, Z. et al. Recapitulating cross-species transmission of simian immunodeficiency virus SIVcpz to humans by using humanized BLT mice. J. Virol. 90, 7728-7739 (2016).
57. Yuan, Z., Kang, G., Daharsh, L., Fan, W. & Li, Q. SIVcpz closely related to the ancestral HIV-1 is less or non-pathogenic to humans in a hu-BLT mouse model. Emerg. Microbes Infect. 7, 59 (2018).
58. Yuan, Z. et al. Human galectin-9 promotes the expansion of HIV reservoirs in vivo in humanized mice. AIDS 37, 571-577 (2023).
59. Aryee, K. et al. Enhanced development of functional human NK cells in NOD-scid-IL2rgnull mice expressing human IL15. FASEB J. 36, e22476 (2022).
60. Yuan, Z. et al. Controlling multicycle replication of live-attenuated HIV-1 using an unnatural genetic switch. ACS Synth. Biol. 6, 721-731 (2017).

That which is claimed:

1. A single chain diabody that binds human immunodeficiency virus 1 Envelope protein variable loops 1 and 2 (HIV-1 Env V1/V2) and human type III Fc receptor (CD16) comprising:
(a) an anti-HIV-1 Env V1/V2 antigen binding fragment comprising a VH comprising or consisting of SEQ ID NO:1;
(b) an anti-HIV Env V1/V2 antigen binding fragment comprising a VL comprising or consisting of SEQ ID NO:5;
(c) an anti-CD16 antigen binding fragment comprising a VH comprising or consisting of SEQ ID NO:17; and
(d) an anti-CD16 antigen binding fragment comprising a VL comprising or consisting of SEQ ID NO:21.

2. A single-chain diabody that binds HIV-1 Env V1/V2 and CD16 comprising:
(a) an anti-HIV-1 Env V1/V2 antigen-binding fragment comprising a VH comprising CDRs 1, 2 and 3, wherein VH CDR1 comprises or consists of SEQ ID NO:2, VH CDR2 comprises or consists of SEQ ID NO:3, and VH CDR3 comprises or consists of SEQ ID NO:4;
(b) an anti-HIV-1 Env V1/V2 antigen-binding fragment comprising a VL comprising CDRs 1, 2 and 3, wherein VL CDR1 comprises or consists of SEQ ID NO:6, VL CDR2 comprises or consists of SEQ ID NO:7, and VL CDR3 comprises or consists of SEQ ID NO:8;
(c) an anti-CD16 antigen-binding fragment comprising a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and
(d) an anti-CD16 antigen-binding fragment comprising a VL comprising CDRs 1, 2 and 3, wherein VL CDR1 comprises or consists of SEQ ID NO:22, VL CDR2 comprises or consists of SEQ ID NO:23, and VL CDR3 comprises or consists of SEQ ID NO:24.

3. A single chain diabody that binds HIV-1 CD4bs and CD16 comprising:
(a) an anti-HIV-1 CD4bs antigen binding fragment comprising a VH comprising or consisting of SEQ ID NO:9;
(b) an anti-HIV CD4bs antigen binding fragment comprising a VL comprising or consisting of SEQ ID NO:13;
(c) an anti-CD16 antigen binding fragment comprising a VH comprising or consisting of SEQ ID NO:17; and
(d) an anti-CD16 antigen binding fragment comprising a VL comprising or consisting of SEQ ID NO:21.

4. A single chain diabody that binds HIV-1 CD4bs and CD16 comprising:
(a) an anti-HIV-1 CD4bs antigen-binding fragment comprising a VH comprising CDRs 1, 2 and 3, wherein VH CDR1 comprises or consists of SEQ ID NO:10, VH CDR2 comprises or consists of SEQ ID NO:11, and VH CDR3 comprises or consists of SEQ ID NO:12;
(b) an anti-HIV-1 CD4bs antigen-binding fragment comprising a VL comprising CDRs 1, 2 and 3, wherein VL CDR1 comprises or consists of SEQ ID NO:14, VL CDR2 comprises or consists of SEQ ID NO:15, and VL CDR3 comprises or consists of SEQ ID NO:16;
(c) an anti-CD16 antigen-binding fragment comprising a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and
(d) an anti-CD16 antigen-binding fragment comprising a VL comprising CDRs 1, 2 and 3, wherein VL CDR1 comprises or consists of SEQ ID NO:22, VL CDR2 comprises or consists of SEQ ID NO:23, and VL CDR3 comprises or consists of SEQ ID NO:24.

5. A bispecific antibody or antigen-binding fragment thereof that specifically binds HIV-1 Env V1/V2 and CD16 comprising:
(a) a first antibody or antigen-binding fragment thereof that specifically binds HIV-1 Env V1/V2 comprising a VH comprising or consisting of SEQ ID NO:1 and a VL comprising or consisting of SEQ ID NO:5; and
(b) a second antibody or antigen-binding fragment thereof that specifically binds CD16 comprising a VH comprising or consisting of SEQ ID NO:17 and a VL comprising or consisting of SEQ ID NO:21.

6. A bispecific antibody or antigen-binding fragment thereof that specifically binds HIV-1 Env V1/V2 and CD16 comprising:
(a) a first antibody or antigen-binding fragment thereof that specifically binds HIV-1 Env V1/V2 comprising (i) a heavy chain variable region (VH) comprising complementarity determining regions (CDRs) 1, 2 and 3, wherein VH CDR1 comprises or consists of SEQ ID NO:2, VH CDR2 comprises or consists of SEQ ID NO:3, and VH CDR3 comprises or consists of SEQ ID NO:4; and (ii) a light chain variable region (VL) comprising CDRs 1, 2 and 3, wherein VL CDR1 comprises or consists of SEQ ID NO:6, VL CDR2 comprises or consists of SEQ ID NO:7, and VL CDR comprises or consists of SEQ ID NO:8; and
(b) a second antibody or antigen-binding fragment thereof that specifically binds CD16 comprising i) a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and (ii) a VL comprising CDRs 1, 2 and 3, wherein VL CDR1 comprises or consists of SEQ ID NO:22, VL CDR2 comprises or consists of SEQ ID NO:23, and VL CDR comprises or consists of SEQ ID NO:24.

7. A bispecific antibody or antigen-binding fragment thereof that specifically binds HIV-1 Env CD4bs and CD16 comprising:
(a) a first antibody or antigen-binding fragment thereof that specifically binds HIV-1 CD4bs comprising a VH comprising or consisting of SEQ ID NO:9 and a VL comprising or consisting of SEQ ID NO:13; and
(b) a second antibody or antigen-binding fragment thereof that specifically binds CD16 comprising a VH comprising or consisting of SEQ ID NO:17 and a VL comprising or consisting of SEQ ID NO:21.

8. A bispecific antibody or antigen-binding fragment thereof that specifically binds HIV-1 CD4 binding site (CD4bs) and CD16 comprising:
(a) a first antibody or antigen-binding fragment thereof that specifically binds HIV-1 CD4bs comprising (i) VH comprising CDRs 1, 2 and 3, wherein VH CDR1 comprises or consists of SEQ ID NO:10, VH CDR2 comprises or consists of SEQ ID NO:11, and VH CDR3 comprises or consists of SEQ ID NO:12; and (ii) a VL comprising CDRs 1, 2 and 3, wherein VL CDR1 comprises or consists of SEQ ID NO:14, VL CDR2 comprises or consists of SEQ ID NO:15, and VL CDR comprises or consists of SEQ ID NO:16; and
(b) a second antibody or antigen-binding fragment thereof that specifically binds CD16 comprising i) a VH comprising CDRs 1, 2 and 3, wherein VH CDR1 comprises or consists of SEQ ID NO:18, VH CDR2 comprises or consists of SEQ ID NO:19, and VH CDR3 comprises or consists of SEQ ID NO:20; and (ii) a VL comprising CDRs 1, 2 and 3, wherein VL CDR1 comprises or consists of SEQ ID NO:22, VL CDR2 comprises or consists of SEQ ID NO:23, and VL CDR comprises or consists of SEQ ID NO:24.

9. A bispecific antibody or antibody-binding fragment thereof that specifically binds HIV-1 Env V1/V2 and CD16 comprising:
(a) a first antibody or antigen-binding fragment thereof that specifically binds HIV-1 Env V1/V2 comprising (i) VH comprising CDRs 1, 2 and 3, wherein VH CDR1 comprises or consists of SEQ ID NO:2, or SEQ ID NO:2 with a substitution at five or fewer amino acid positions, VH CDR2 comprises or consists of SEQ ID NO:3, or SEQ ID NO:3 with a substitution at seven or fewer amino acid positions, and VH CDR3 comprises or consists of SEQ ID NO:4, or SEQ ID NO:4 with a substitution at fifteen or fewer amino acid positions; and (ii) a VL comprising CDRs 1, 2 and 3, wherein VL CDR1 comprises or consists of SEQ ID NO:6, or SEQ ID NO:6 with a substitution at seven or fewer amino acid positions, VH CDR2 comprises or consists of SEQ ID NO:7, or SEQ ID NO:7 with a substitution at four or fewer amino acid positions, and VH CDR3 comprises or consists of SEQ ID NO:8, or SEQ ID NO:8 with a substitution at five or fewer amino acid positions; and
(b) a second antibody or antigen-binding fragment thereof that specifically binds CD16 comprising (i) VH comprising CDRs 1, 2 and 3, wherein VH CDR1 comprises or consists of SEQ ID NO:18, or SEQ ID NO:18 with a substitution at three or fewer amino acid positions, VH CDR2 comprises or consists of SEQ ID NO:19, or SEQ ID NO:19 with a substitution at seven or fewer amino acid positions, and VH CDR3 comprises or consists of SEQ ID NO:20, or SEQ ID NO:20 with a substitution at four or fewer amino acid positions; and (ii) a VL comprising CDRs 1, 2 and 3, wherein VL CDR1 comprises or consists of SEQ ID NO:22, or SEQ ID NO:22 with a substitution at six or fewer amino acid positions, VH CDR2 comprises or consists of SEQ ID NO:23, or SEQ ID NO:23 with a substitution at four or fewer amino acid positions, and VH CDR3 comprises or consists of SEQ ID NO:24, or SEQ ID NO:24 with a substitution at six or fewer amino acid positions.

10. A bispecific antibody or antibody-binding fragment thereof that specifically binds HIV-1 CD4bs and CD16 comprising:
(a) a first antigen-binding fragment thereof that specifically binds HIV-1 Env V1/V2 comprising (i) VH comprising CDRs 1, 2 and 3, wherein VH CDR1 comprises or consists of SEQ ID NO:10, or SEQ ID NO:10 with a substitution at three or fewer amino acid positions, VH CDR2 comprises or consists of SEQ ID NO:11, or SEQ ID NO:11 with a substitution at nine or fewer amino acid positions, and VH CDR3 comprises or consists of SEQ ID NO:12, or SEQ ID NO:12 with a substitution at five or fewer amino acid positions; and (ii) a VL comprising CDRs 1, 2 and 3, wherein VL CDR1 comprises or consists of SEQ ID NO:14, or SEQ ID NO:14 with a substitution at four or fewer amino acid positions, VH CDR2 comprises or consists of SEQ ID NO:15, or SEQ ID NO:15 with a substitution at four or fewer amino acid positions, and VH CDR3 comprises or consists of SEQ ID NO:16, or SEQ ID NO:16 with a substitution at three or fewer amino acid positions; and
(b) a second antigen-binding fragment thereof that specifically binds CD16 comprising (i) VH comprising CDRs 1, 2 and 3, wherein VH CDR1 comprises or consists of SEQ ID NO:18, or SEQ ID NO:18 with a substitution at three or fewer amino acid positions, VH CDR2 comprises or consists of SEQ ID NO:19, or SEQ ID NO:19 with a substitution at seven or fewer amino acid positions, and VH CDR3 comprises or consists of SEQ ID NO:20, or SEQ ID NO:20 with a substitution at four or fewer amino acid positions; and (ii) a VL comprising CDRs 1, 2 and 3, wherein VL CDR1 comprises or consists of SEQ ID NO:22, or SEQ ID NO:22 with a substitution at six or fewer amino acid positions, VH CDR2 comprises or consists of SEQ ID NO:23, or SEQ ID NO:23 with a substitution at four or fewer amino acid positions, and VH CDR3 comprises or consists of SEQ ID NO:24, or SEQ ID NO:24 with a substitution at six or fewer amino acid positions.

11. A bispecific antibody or antigen-binding fragment thereof that specifically binds HIV-1 Env and CD16.

12. The bispecific antibody or antigen-binding fragment thereof of claim 11, wherein the antibody or antigen-binding fragment specifically binds HIV-1 Env V1/V2.

13. The bispecific antibody or antigen-binding fragment thereof of claim 11, wherein the antibody or antigen-binding fragment specifically binds HIV-1 CD4bs.

14. The bispecific antibody or antigen-binding fragment thereof of claim 12, wherein the antibody or antigen-binding fragment thereof that specifically binds HIV-1 Env V1/V2 comprises a VH having at least 90% sequence identity to SEQ ID NO:2.

15. The bispecific antibody or antigen-binding fragment thereof of claim 12, wherein the antibody or antigen-binding fragment thereof that specifically binds HIV-1 Env V1/V2 comprises a VL having at least 90% sequence identity to SEQ ID NO:5.

16. The bispecific antibody or antigen-binding fragment thereof of claim 13, wherein the antibody or antigen-binding fragment thereof that specifically binds HIV-1 CD4bs comprises a VH having at least 90% sequence identity to SEQ ID NO:9.

17. The bispecific antibody or antigen-binding fragment thereof of claim 13, wherein the antibody or antigen-binding fragment thereof that specifically binds HIV-1 CD4bs comprises a VL having at least 90% sequence identity to SEQ ID NO:5.

18. The bispecific antibody or antigen-binding fragment thereof of claim 12 or 13, wherein the antibody or antigen-binding fragment thereof that specifically binds CD16 comprises a VH having at least 90% sequence identity to SEQ ID NO:17.

19. The bispecific antibody or antigen-binding fragment thereof of claim 12 or 13, wherein the antibody or antigen-binding fragment thereof that specifically binds CD16 comprises a VL having at least 90% sequence identity to SEQ ID NO:21.

20. A method for treating HIV-1 in a patient comprising the step of administering to the patient an effective amount of a single chain diabody of any of claims 1-4 or a bispecific antibody or antigen-binding fragment thereof of any of claims 5-19.

21. The bispecific antibody or antigen-binding fragment thereof of any of claims 5-20, wherein the bispecific antibody or antigen-binding fragment thereof comprises tandem scFvs, diabody format, single-chain diabodies, tandem diabodies (TandAbs), dual-affinity retargeting molecules (DARTs), dock-and-lock (DNL), and nanobodies.


SEQUENCE LISTING (28 sequences)

SEQ ID NO: 1 - protein, 138 aa, unidentified
QEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSH

SEQ ID NO: 2 - protein, 11 aa, unidentified
SGFTFHKYGMH

SEQ ID NO: 3 - protein, 15 aa, unidentified
ISDDGMRKYHSDSMW

SEQ ID NO: 4 - protein, 28 aa, unidentified
EAGGPIWHDDVKYYDFNDGYYNYHYMDV

SEQ ID NO: 5 - protein, 110 aa, unidentified
QSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVL

SEQ ID NO: 6 - protein, 14 aa, unidentified
NGTSSDVGGFDSVS

SEQ ID NO: 7 - protein, 8 aa, unidentified
DVSHRPSG

SEQ ID NO: 8 - protein, 10 aa, unidentified
SSLTDRSHRI

SEQ ID NO: 9 - protein, 123 aa, unidentified
QVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSS

SEQ ID NO: 10 - protein, 5 aa, unidentified
DYFIH

SEQ ID NO: 11 - protein, 17 aa, unidentified
WINPKTGQPNNPRQFQG

SEQ ID NO: 12 - protein, 10 aa, unidentified
QRSDYWDFDV

SEQ ID NO: 13 - protein, 99 aa, unidentified
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLK

SEQ ID NO: 14 - protein, 7 aa, unidentified
QANGYLN

SEQ ID NO: 15 - protein, 7 aa, unidentified
DGSKLER

SEQ ID NO: 16 - protein, 5 aa, unidentified
QVYEF

SEQ ID NO: 17 - protein, 117 aa, unidentified
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 18 - protein, 5 aa, unidentified
DYGMS

SEQ ID NO: 19 - protein, 17 aa, unidentified
GINWNGGSTGYADSVKG

SEQ ID NO: 20 - protein, 8 aa, unidentified
GRSLLFDY

SEQ ID NO: 21 - protein, 108 aa, unidentified
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 22 - protein, 11 aa, unidentified
QGDSLRSYYAS

SEQ ID NO: 23 - protein, 6 aa, unidentified
GKNNRP

SEQ ID NO: 24 - protein, 11 aa, unidentified
NSRDSSGNHVV

SEQ ID NO: 25 - protein, 122 aa, unidentified
EVQLVESGGGLVQPGGSLRLSCAASGFNVYASGMHWVRAQAPGKGLEWVAKIYPDSDYTYYADSVKGRFTISADTSKNTAYLQMNSLRAEDTAVYYCSRDSSFYYVYAMDYWGQGTLVTVSS

SEQ ID NO: 26 - protein, 108 aa, unidentified
DIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIK

SEQ ID NO: 27 - protein, 15 aa, synthetic construct
GGGGSGGGGSGGGGS

SEQ ID NO: 28 - protein, 10 aa, synthetic construct
GGGGSGGGGS